(19) United States
(12) Patent Application Publication
(10) Pub. No.: US 2023/0090439 A1
(43) Pub. Date: Mar. 23, 2023

(54) PHARMACEUTICAL COMPOSITION FOR CONTROLLING PARASITES ON NON-HUMAN ORGANISMS

(71) Applicant: Bayer Animal Health GmbH, Leverkusen (DE)

(72) Inventors: Anne FROEHLICH, Leverkusen (DE); Andreas TURBERG, Haan (DE); Sandra MENSINGER, Koeln (DE); Daris GRIZIC, Duesseldorf (DE); Daniel GONDOL, Leichlingen (DE); William BARTON, Forest City, MO (US)

(21) Appl. No.: 17/795,652

(22) PCT Filed: Jan. 27, 2021
(86) PCT No.: PCT/EP2021/051891

§ 371 Date: Jul. 27, 2022

Related U.S. Application Data
(60) Provisional application No. 62/967,281, filed on Jan. 29, 2020.

(51) Int. Cl. A61K 31/4439; A61K 9/08; A61K 31/415; A61K 31/42; A61K 31/402; A61K 9/00; A61P 33/14

(52) U.S. Cl. CPC A61K 31/4439; A61K 9/08; A61K 31/415; A61K 31/42; A61K 31/402; A61K 9/0017; A61P 33/14

(57) ABSTRACT

The present invention relates to a pharmaceutical composition comprising at least one pharmaceutically active ingredient, which is completely soluble in a solvent phase, characterized in that the pharmaceutically active ingredient is in particular from the group of fluorinated heteroarylamides, and that the solvent phase is configured as a solvent mixture and comprises at least one first solvent and a second solvent different from the first solvent, wherein the first solvent comprises a C1 to C15-alcohol, wherein the second solvent comprises an organic carbonate, and also wherein the first solvent is present in a proportion from 50% by weight to 95% by weight and wherein the second solvent is present in a proportion from 5% by weight to 50% by weight, based in each case on the sum total of the solvents in the solvent phase.

The present invention relates to a pharmaceutical composition for controlling parasites on non-human organisms. The present invention relates particularly to dermally applicable formulations with improved applicability to the organism for controlling ectoparasites on mammals and livestock.

Modern fluorine-containing antiparasitic active compounds, also referred to as fluorinated antiparasitics, for example fluralaner, afoxolaner, sarolaner, lotilaner, fipronil, which has been known for a relatively long time, and the active ingredients described in WO2015/067646 and WO2015/067647 have good insecticidal and acaricidal efficacy. However, many of these active compounds have very low solubility in solvents suitable for topical administration to animals. To avoid the formulation dripping off from the coat after administration, or incomplete distribution on the animal skin, the active compound should preferably be dissolved in a low volume of solvent. Furthermore, the solvent must be toxicologically safe and have high skin compatibility.

WO96/17520 refers to formulations, which are applied dermally to animals for controlling parasitic insects, having the following composition: agonists or antagonists of the nicotinic acetylcholine receptors of insects at a concentration of 1 to 20% by weight, based on the total weight of the formulation; solvents of the benzyl alcohol group or optionally pyrrolidones at a concentration of at least 20% by weight, based on the total weight of the formulation; optionally other solvents from the group of cyclic carbonates or lactones at a concentration of 5.0 to 80% by weight, based on the total weight of the formulation; optionally other auxiliaries from the group of thickeners, spreaders, dyes, antioxidants, blowing agents, preservatives, adhesives, emulsifiers at a concentration of 0.025 to 10% by weight, based on the total weight of the formulation.

Externally applicable pour-on products against parasites in animals are known, the formulation of which comprises isopropanol as solvent. These particularly include products comprising the active ingredient ivermectin, for example Baymec® Pour-on (Bayer®), Ivomec® Pour-on (Merial®) and others. Examples of pour-on products comprising isopropanol with other active ingredients are: Sebacil® Pour-on (Bayer®, active ingredient Phoxim), Dectomax® (Elanco, active ingredient: Doramectin).

The object of the present invention is the provision of a pharmaceutical composition for topical application to non-human organisms using a solvent in which modern, for example fluorinated antiparasitics, especially from the group of heteroarylamides, have a sufficiently high solubility and at the same time good applicability to the animal, especially as pour-on formulations.

A pharmaceutical composition is provided, comprising at least one parasiticidal pharmaceutically active ingredient, and especially easy to apply to animals, which is completely soluble in a solvent phase, wherein the pharmaceutically active ingredient is in particular from the group of fluorinated heteroarylamides, preferably pyridyl amides, and wherein the solvent phase is configured as a solvent mixture and comprises a first solvent and a second solvent different from the first solvent, wherein the first solvent comprises a C1 to C15-alcohol, wherein the second solvent comprises an organic carbonate, and wherein the first solvent is present in a proportion from 50% by weight to 95% by weight and wherein the second solvent is present in a proportion from 5% by weight to 50% by weight, based in each case on the sum total of the solvents in the solvent phase.

For example, the first solvent and the second solvent can be in a quantitative ratio to each other from 50% by weight:50% by weight to 95% by weight:5% by weight, based on the sum total of the solvents in the solvent phase.

According to a further embodiment, the first solvent may comprise only one solvent or a combination of two or more, preferably two C1 to C15-alcohols, wherein in this configuration, the majority of C1 to C15-alcohols are present together in the aforementioned amount and are referred to together as the first solvent. Therefore, in the context of the invention, a solvent is not necessarily only understood to mean one substance but a solvent can also be understood to mean a mixture of different substances. However, it is intended that the substances forming the first solvent are all C1 to C15-alcohols.

Alternatively or additionally, it may be provided that the second solvent consists of one substance or may be a combination of two or more, preferably two substances, wherein the substances forming the second solvent are all organic carbonates. In this configuration, the two or more organic carbonates are present together in the aforementioned amount.

A solvent phase consisting of only the first solvent and the second solvent is also referred to as a binary solvent phase.

A composition according to the invention particularly enables the provision of novel liquid medicaments for topical application or dermal application to non-human organisms using a solvent in which particularly fluorinated antiparasitics from the group of heteroarylamides have a sufficiently high solubility and at the same time good applicability to the animal, especially as pour-on formulations.

The pharmaceutical composition described here also comprises at least one pharmaceutically active ingredient hereinafter also called active ingredient, which may also be referred to as API (active pharmaceutical ingredient). In particular, the active ingredient can be the actual active substance in the composition and therefore mainly or wholly gives rise to the antiparasitic effect, which is described in detail below. Accordingly, the active ingredient should be effective particularly in non-human organisms.

In principle, the pharmaceutically active ingredient is not limited. However, it is provided that the pharmaceutically active ingredient is made up of the group of in particular fluorinated heteroarylamides, which are described in greater detail below. In principle, the heteroarylamide can be, for example, a pyridyl amide, which is a pyridine group bonded via an aryl group to the amide. For example, the heteroarylamide may be fluorinated.

With respect to the pharmaceutically active ingredient, it is further provided that this is fully soluble in a solvent phase. This enables in particular an advantageous dermal applicability, wherein in particular a pour-on application or a solution for pouring on is possible.

A pour-on application, where a spot-on formulation can be an embodiment, is understood to mean in particular an application of the type in which relatively small amounts of formulation are applied, for example up to 100 mL/100 kg, approximately in a range from 1 mL/100 kg to 100 mL/100 kg, for example in a range from 1 mL/100 kg to 20 mL/100 kg, approximately in a range from 5 mL/100 kg to 10 mL/100 kg, based on the amount of formulation applied to the weight of the organism, to confined skin areas, typically on the necks or backs of the non-human organism. Accordingly, a pour-on formulation is understood to mean a formulation of the kind which is suitable and is intended to be applied as a pour-on application.

Particularly important for such applications, i.e. in so-called pour-on solutions, is that a high level of solubility for the active ingredient is achieved which enables a desired concentration of the active ingredient in the solution. Moreover, good spreading characteristics on skin should be achieved. Spreading characteristics can be understood to mean in particular the distribution of the formulation on the skin. Therefore, through the spreading characteristics, it can be determined whether the formulation present as a solution attains a desired distribution on the organism and in particular does not drip off or run off to an excessive extent from the application position such that a locally confined application is possible and no active ingredient is lost by run-off after application.

In order to enable the aforementioned advantageous properties, especially for pour-on formulations, it is intended in the composition described here that the solvent phase is configured as a solvent mixture and comprises a first solvent and a second solvent different from the first solvent, wherein the first solvent comprises a C1 to C15-alcohol, wherein the second solvent is an organic carbonate, and also wherein the first solvent is present in a proportion from 50% by weight to 95% by weight and wherein the second solvent is present in a proportion from 5% by weight to 50% by weight, based in each case on the sum total of the solvents in the solvent phase, in which in a binary solvent phase the first solvent and the second solvent are present in a quantitative ratio to each other of from 50% by weight:50% by weight to 95% by weight:5% by weight, based on the sum total of the solvents in the solvent phase. However, it should be noted that the sum total of the proportions of the first and of the second solvent do not necessarily have to add up to 100% by weight since, for example, also ternary systems with further solvents are conceivable and may be preferred.

In one embodiment, such a configuration can advantageously provide a sufficient concentration of completely dissolved active ingredient of a heteroarylamide in the solvent phase and is thus possible in the formulation and also advantageous spreading characteristics are achievable for a pour-on application. In one embodiment, the active heteroarylamide is at least 95% by weight (of the active) dissolved. In another embodiment, the active is at least 97% by weight (of the active) dissolved. In a further embodiment, the active is 99% by weight (of the active) dissolved. In a preferred embodiment, the active is 100 by weight (of the active) % dissolved.

Thus, in contrast to the assumptions made in the prior art, it has been shown that with a solvent mixture comprising a C1 to C15-alcohol, especially a C1 to C10-alcohol, for example a C1 to C7-alcohol, and an organic carbonate, positive properties can be obtained. For example, the solvent mixture may consist of one or more C1 to C15-alcohols and one or more organic carbonates. These show in combination, for example, good solubility of an active ingredient, particularly formed from the group of fluorinated heteroarylamides, and good spreading characteristics.

C1 to C15-alcohols are in principle understood to mean aliphatic or aromatic alcohols having 1 to 15 carbon atoms.

Organic carbonates are also understood to mean acyclic or cyclic carbonic esters which are polar solvents of low volatility per se, which are characterized by their low toxicity. By skillful selection of the alcohols for the ester synthesis, the physical properties of the resulting carbonates can be adjusted to the desired field of application.

Thus, the present invention provides a highly suitable composition for pour-on application, both with regard to the spreading characteristics, the facility of administration, and with respect to the active ingredient concentration, i.e. the efficacy.

Further advantages of the formulation described here are good compatibility and safety for the treated organism, such as reduced harmful effects on the animal, for example on the skin and/or the coat, such as irritation or residues in the body of the animal, including in animals entering the food chain. The formulations and the ease of administration (good applicability due to viscosity and spreading) provide excellent environmental sustainability, such that high acceptance by the consumer is achieved and harmful side effects are reduced. This applies even in the case of use on livestock in food production. Furthermore, the formulation permits the active ingredient present, by means of the increased dissolved concentrations, to provide high efficacy in less time in order to be effective and therefore provides a sustained active period which obviates the need for frequent application. Finally, good handling and user friendliness is achieved with concomitant low costs for the manufacturing of the formulation. Also advantageous is the good bioavailability and spectrum of use.

Furthermore, a composition of the invention can be readily applicable to the animal, for instance with regard to the viscosity and spreading characteristics. The composition, especially the active ingredient, also has a low tendency to be washed off, for example by rain. In fact, the efficacy can be maintained even in the case of wet animals or irradiation from the sun. The compositions generally possess high stability and exhibits reduced degradation and reduced secondary reactions of components of the composition, for instance with each other. Preferred stability which is achieved by the composition described here, includes a shelf-life of at least 12 months, preferably 24 months, at 30° C. After application, the composition readily dries off, especially by the addition of a good evaporating solvent, without significantly impairing the appearance of the animal.

With respect to the solvent present in the solvent phase or in the solvent mixture, the following solvents are particularly advantageous. The first solvent is different from the second solvent. In one embodiment, the first solvent, as distinct from the second solvent, is not a carbonate.

In one embodiment, the first solvent preferably comprises C1-C₄-alkanols which are optionally substituted by at least one C1-C₄-alkoxy group or phenyl group, wherein the hydroxyl group can in principle be in any selectable position. In a specific embodiment, the first solvent is selected from the group consisting of ethanol, n-propanol, isopropanol, butanol, for example isobutanol or n-butanol, especially ethanol and/or isopropanol. In addition to the good dissolution properties, particularly in connection with the organic carbonates described, such solvents offer the further advantage of suitable toxicities for use in non-human organisms, such that application is possible without adverse effect in the animal.

The second solvent is, preferably an organic aliphatic C1-C₄-carbonate, optionally substituted with an hydroxyl group. In a specific embodiment, the second solvent is selected from the group consisting of ethylene carbonate, propylene carbonate, glycerol carbonate. In addition to the good dissolution properties, particularly in connection with the alcohols described, such solvents offer the further advantage of suitable toxicities for use in non-human organisms, such that application is possible without adverse effects in the animal.

In a particularly preferred embodiment, the first solvent is isopropanol and the second solvent is propylene carbonate. It has been found that a solvent phase comprising isopropanol and propylene carbonate as solvent, for example provides the aforementioned advantages with respect to good solubility of the active ingredient, advantageous spreading characteristics, and at the same time, possesses a lower toxicity.

In another embodiment, the solvent mixture consists of the first solvent, i.e. one or more first solvents as described above, and the second solvent, i.e. one or more second solvents as described above. In other words, the aforementioned first solvent and second solvent may be sufficient exclusively as solvent, in which in a comprehensible manner, one of the first solvents described and one of the second solvents described may be sufficient to form the solvent mixture.

In one embodiment, with respect to the relative ratio of the first solvent to second solvent, it is preferred that the second solvent, i.e. the organic carbonate, is present at a content of ≥20% by weight to ≤45% by weight, preferably at a content of ≥30% by weight to ≤40% by weight, based on the sum total of the solvents in the solvent mixture. In this embodiment, the first solvent is present at a content of ≥55% by weight to ≤80% by weight, preferably at a content of ≥60% by weight to ≤70% by weight, based on the sum total of the solvents in the solvent mixture. In this configuration, the aforementioned advantages with respect to the solubility of the active ingredient, with concomitant good spreading characteristics, can be particularly effectively provided. In other words, with consistent results, exceptional solubility of the active ingredient can be facilitated. Therefore, advantageous applicability, along with stable storage characteristics is provided together. Because of the high solubility of the active ingredient, comparatively low amounts of the solvent mixture are needed. As a result, the composition can be stored.

Furthermore, in another embodiment the solvent mixture, in addition to the first solvent and the second solvent, comprises a third solvent different from the first solvent and the second solvent. Therefore, the third solvent is not designed as has been described above for the first solvent and the second solvent.

By using a further solvent, the solubility and/or the spreading characteristics can be potentially further improved or at least can be adjusted to the desired use.

In a specific embodiment, the solvent phase is configured, as a mixture of at least three solvents, in which the third solvent is neither a C1 to C15-alcohol nor an organic carbonate, but rather has a hereto different definition. However, this does not preclude that the first solvent and/or the second solvent and/or the third solvent is present in each case as a binary solvent mixture for example.

In principle, by providing a ternary solvent phase, the first solvent and the second solvent can be present in the range described above, in which the proportion of the first solvent and the second solvent may be comprised of a proportion of the third solvent. In particular, in an embodiment the third solvent substitutes a part of the first solvent, in which the first solvent is still present in the described range of 50% by weight to 95% by weight, based on the sum total of the solvents in the solvent phase, and in which the second solvent is likewise also present in the quantitative range described above, namely at a proportion of 5% by weight to 50% by weight, based in each case on the sum total of the solvent in the solvent phase. The third solvent, as well as the first solvent and the second solvent, should be pharmaceutically compatible and also not influence the solubility of the active ingredients unfavorably. It may be useful to use penetration-enhancing solvents.

Advantageous examples of the third solvent, which is different from the first solvent and the second solvent, are selected from the group consisting of water, butylhydroxytoluene, N-methylpyrrolidone, 2-pyrrolidone, dimethyl sulfoxide, triethyl phosphate, benzyl benzoate, octyldodecanol, paraffin, triglycerides such as caprylic/capric acid triglycerides (e.g. Miglyol 812), propylene glycol octanoate decanoate (e.g. Miglyol 840), glycol ethers such as diethylene glycol methyl ether, diethylene glycol monoethyl ether, dipropylene glycol monoethyl ether, diethylene glycol monobutyl ether.

In one embodiment, the solvent phase can be a ternary system and therefore consist of the first solvent or the first solvents, the second solvent or the second solvents and the third solvent or the third solvents. In this embodiment, the first solvent(s), the second solvent(s) and the third solvent(s) are selectable as defined above.

In particular, in the embodiment of a ternary system, one non-limiting example is that the sum total of first and third solvent and the second solvent are present in a quantitative ratio to each other of from 50% by weight:50% by weight to 95% by weight:5% by weight, based on the sum total of the solvents in the solvent phase. With respect to the relative ratio of the first solvent to second solvent, it is preferred that the second solvent is present at a content of ≥20% by weight to ≤45% by weight, preferably at a content of ≥30% by weight to ≤40% by weight, based on the sum total of the solvents in the solvent mixture. Accordingly, the first solvent and optionally the third solvent can be together present at a content of ≥55% by weight to ≤80% by weight, preferably at a content of ≥60% by weight to ≤70% by weight, based on the sum total of the solvents in the solvent mixture.

Non-limiting examples of solvent mixtures can be configured, for example, as follows: A mixture of, isopropanol (32.5), ethanol (32.5) and propylene carbonate (35) may be used, wherein the numbers in brackets are intended to be the content in % by weight, based on the solvent mixture. As further examples, a mixture of isopropanol (32.5), ethanol (32.5) and glycerol carbonate (35), isopropanol (65), propylene carbonate (17.5) and glycerol carbonate (17.5) or also a mixture of ethanol (65), propylene carbonate (17.5) and glycerol carbonate (17.5) can be used, wherein the numbers in brackets in turn are intended to be the content in % by weight, based on the solvent mixture.

In one embodiment one first solvent and one solvent mixture composed of, for example, two second solvents and optionally one or more third solvents are present. In another embodiment one second solvent and a solvent mixture composed of, for example, two first solvents and optionally one or more third solvents are present.

In addition, the constituents of the composition can be restricted to the solvent and the active ingredient such that the composition may consist of the first solvent, the second solvent, optionally the third solvent and the active ingredient. However, further constituents are not intended to be excluded and are defined elsewhere.

Preferably, the fluorinated heteroarylamide is designed as described in WO 2015067646 A1 or WO 2015067647 A1. Active ingredients of this kind are preferably configured as follows and comprise compounds of the general formula (I):

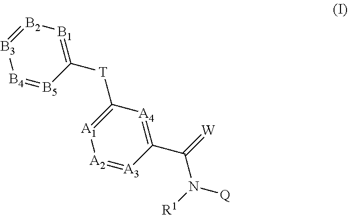

in which
R¹ is H, in each case optionally substituted C₂-C₆-alkenyl, C₂-C₆-alkynyl, C₃-C₇-cycloalkyl, C₁-C₆-alkylcarbonyl, C₁-C₆-alkoxycarbonyl, aryl-(C₁-C₃)-alkyl, heteroaryl-(C₁-C₃)-alkyl or is optionally substituted C₁-C₆-alkyl, preferably H or preferably C₁-C₂-alkyl, very particularly preferably H or methyl, in particular H, the moieties are as follows:
A₁ is CR² or N,
A₂ is CR³ or N,
A₃ is CR⁴ or N,
A₄ is CR⁵ or N,
B₁ is CR⁶ or N,
B₂ is CR⁷ or N,
B₃ is CR⁸ or N,
B₄ is CR⁹ or N, and
B₅ is CR′ or N, but not more than three of the A₁ to A₄ moieties are N and not more than three of the B₁ to B₅ moieties are N;
R², R³, R⁴, R⁵, R⁶, R⁷, R⁹ and R¹⁹ are each independently of one another H, halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, N—C₁-C₆-alkylamino, N,N-di-C₁-C₆-alkylamino or N—C₁-C₃-alkoxy-C₁-C₄-alkylamino or 1-pyrrolidinyl;
if neither of the A₂ and A₃ moieties is N, R³ and R⁴ together with the carbon atom to which they are bonded may form a 5- or 6-membered ring containing 0, 1 or 2 nitrogen atoms and/or 0 or 1 oxygen atom and/or 0 or 1 sulfur atom; or if neither of the A₁ and A₂ moieties is N, R² and R³ together with the carbon atom to which they are bonded may form a 6-membered ring containing 0, 1 or 2 nitrogen atoms;
R⁸ is halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, N—C₁-C₆-alkylamino or N,N-di-C₁-C₆-alkylamino;
W is O or S,
Q is H, formyl, hydroxyl, amino or in each case optionally substituted C₁-C₆-alkyl, C₂-C₆-alkenyl, C₂-C₆-alkynyl, C₃-C₆-cycloalkyl, C₁-C₅-heterocycloalkyl, C₁-C₄-alkoxy, C₁-C₆-alkyl-C₃-C₆-cycloalkyl, C₃-C₆-cycloalkyl-C₁-C₆-alkyl, C₆-,C₁₀-C₁₄-aryl, C₁-C₅-heteroaryl, C₆-,C₁₀-,C₁₄-aryl-(C₁-C₃)-alkyl, C₁-C₅-heteroaryl-(C₁-C₃)-alkyl, N—C₁-C₄-alkylamino, N—C₁-C₄-alkylcarbonylamino, or N,N-di-C₁-C₄-alkylamino; or is an optionally poly-V-substituted unsaturated 6-membered carbocycle; or is an optionally poly-V-substituted unsaturated 4-, 5- or 6-membered heterocyclic ring, where
V independently of one another is halogen, cyano, nitro, in each case optionally substituted C₁-C₆-alkyl, C₁-C₄-alkenyl, C₁-C₄-alkynyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, or N,N-di-(C₁-C₆-alkyl)amino;
T is an optionally substituted 5-membered heteroaromatic system containing not more than 2 heteroatoms (1 or 2 heteroatoms), such as four carbon atoms and one (1) heteroatom, preferably one (1) nitrogen, one (1) oxygen or one (1) sulfur atom or three carbon atoms and two heteroatoms, preferably two nitrogen atoms, one (1) nitrogen and one (1) oxygen atom, or one (1) nitrogen and one (1) sulfur atom, and salts, N-oxides and tautomeric forms of the compounds of the formula (I).

Described are, for example, compounds of the formula (Ia)

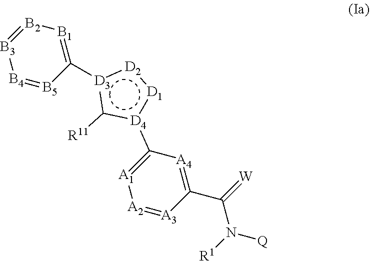

in which
the D₁, D₂ moieties each independently of one another are C—R¹¹ or a heteroatom selected from N and O;
the D₃ and D₄ moieties independently of one another are C or represent a heteroatom selected from N (in other words: the D₃ and D₄ moieties independently of one another represent C or N);
where not more than one (1) or two moieties selected from D₁, D₂, D₃ and D₄ are a heteroatom, where one (1) or two moieties selected from D₁, D₂, D₃ and D₄ are a heteroatom selected from N or O in the case of D₁ and D₂, or N in the case of D₃ and D₄;
[symbol] is an aromatic system; and
R¹, A₁, A₂, A₃, A₄, B₁, B₂, B₃, B₄, B₅, R², R³, R⁴, R⁵, R⁶, R⁷, R⁸, R⁹, R¹⁰, w, Q, V, and T are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N, and salts, N-oxides and tautomeric forms of the compounds of the formula (I).

One embodiment of the present invention relates to compounds of the formula (Ia′)

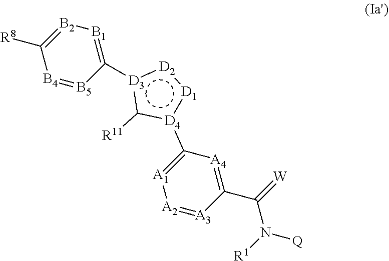

in which

R¹, R¹¹, Q, W, A₁, A₂, A₃, A₄, B₁, B₂, B₄ and B₅ are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N;

D₁ and D₂ each independently of one another are C—R¹¹ or a heteroatom, preferably C—R¹¹ or a heteroatom selected from N, O or S, more preferably C—R¹¹ or a heteroatom selected from N or O;
the D₃ and D₄ moieties independently of one another are C or a heteroatom selected from N;

where not more than one (1) or two moieties selected from D₁, D₂, D₃ and D₄ is/are a heteroatom, where one (1) or two moieties selected from D₁, D₂, D₃ and D₄ are a heteroatom selected from N or O in the case of D₁ and D₂, or N in the case of D₃ and D₄;

[symbol] is an aromatic system,

and R⁸ is as defined herein, preferably is perfluorinated C₁-C₄-alkyl.

A further embodiment of the present invention relates to compounds of the formula (la′)

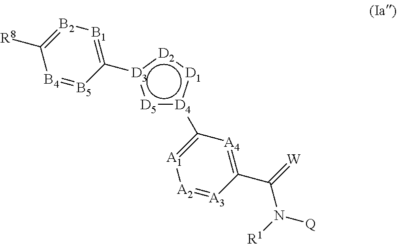

in which
D₁ is C—R¹¹ or a heteroatom selected from N or O;
D₂ is C—R¹¹ or a heteroatom selected from N or O;
D₃ is C or N;
D₄ is C or N;
D₅ is C—R¹¹ or N;
where not more than one (1) or two moieties selected from D₁, D₂, D₃, D₄ and D₅ are a heteroatom;
   [symbol] is an aromatic system; and
   R¹ is H, in each case optionally substituted C₂-C₆-alkenyl, C₂-C₆-alkynyl, C₃-C₇-cycloalkyl, C₁-C₆-alkylcarbonyl, C₁-C₆-alkoxycarbonyl, aryl-(C₁-C₃)-alkyl or heteroaryl-(C₁-C₃)-alkyl or is optionally substituted C₁-C₆-alkyl, particularly preferably H;
the moieties are as follows:
A₁ is CR² or N,
A₂ is CR³ or N,
A₃ is CR⁴ or N,
A₄ is CR⁵ or N,
B₁ is CR⁶ or N,
B₂ is CR⁷ or N,
B₃ is CR⁸ or N,
B₄ is CR⁹ or N, and
B₅ is CR¹⁰ or N,
but not more than three of the A₁ to A₄ moieties are N and not more than three of the B₁ to B₅ moieties simultaneously are N;
R², R³, R⁴, R⁵, R⁶, R⁷, R⁹ and R¹⁹ each independently of one another are H, halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfonyl, N—C₁-C₆-alkylamino or N,N-di-C₁-C₆-alkylamino;

if neither of the A₂ and A₃ moieties is N, R³ and R⁴ together with the carbon atom to which they are bonded may form a 5- or 6-membered ring containing 0, 1 or 2 nitrogen atoms and/or 0 or 1 oxygen atom and/or 0 or 1 sulfur atom, or if neither of the A₁ and A₂ moieties is N, R² and R³ together with the carbon atom to which they are bonded may form a 6-membered ring containing 0, 1 or 2 nitrogen atoms;
R⁸ is halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, alkylsulfanyl, C₁-C₆-alkylsulfonyl, N—C₁-C₆-alkylamino or N,N-di-C₁-C₆-alkylamino;
R¹¹ independently of one another is H, halogen, cyano, nitro, amino or an optionally substituted C₁-C₆-alkyl, C₁-C₆-alkyloxy, C₁-C₆-alkylcarbonyl, C₁-C₆-alkylsulfanyl, C₁-C₆-alkylsulfonyl, preferably H;
W is O or S,
Q is H, formyl, hydroxyl, amino or in each case optionally substituted C₁-C₆-alkyl, C₂-C₆-alkenyl, C₂-C₆-alkynyl, C₃-C₆-cycloalkyl, C₁-C₅-heterocycloalkyl, C₁-C₄-alkoxy, C₁-C₅-heteroaryl, aryl-(C₁-C₃)-alkyl, C₁-C₅-heteroaryl-(C₁-C₃)-alkyl, N—C₁-C₄-alkylamino, N—C₁-C₄-alkylcarbonylamino, or N,N-di-C₁-C₄-alkylamino; or
is an optionally poly-V-substituted unsaturated 6-membered carbocycle; or is an optionally poly-V-substituted unsaturated 4-, 5- or 6-membered heterocyclic ring, where
V independently of one another is halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfonyl, or N,N-di-(C₁-C₆-alkyl)amino;

and salts, N-oxides and tautomeric forms of the compounds of the formula (Ia″).

Further suitable are compounds of the formula (Ia″), where the compounds of the formula (Ia″) are compounds of the formula (I-T3)

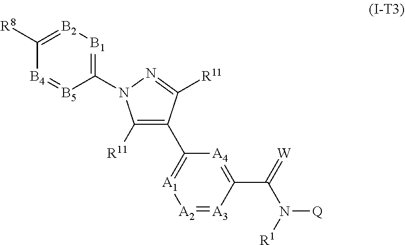

in which R¹, A₁, A₂, A₃, A₄, R¹¹, B₁, B₂, B₄, B₅, R⁸, Q and W are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N.

A further embodiment of the present invention relates to compounds of the formula (Ia″), where the compounds of the formula (Ia″) are compounds of the formula (I-T2)

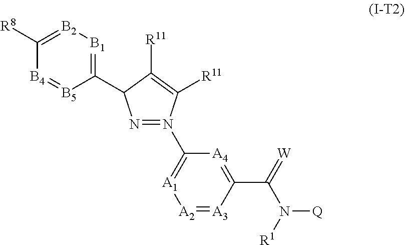

in which R¹, A₁, A₂, A₃, A₄, R¹¹, B₁, B₂, B₄, B₅, R⁸, Q and W are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N.

A further embodiment of the present invention relates to compounds of the formula (Ia″), where the compounds of the formula (Ia″) are compounds of the formula (I-T4)

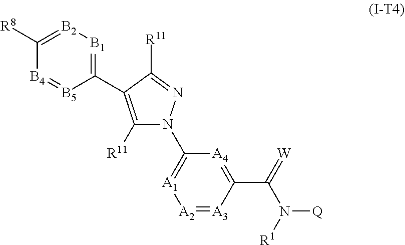

in which R¹, A₁, A₂, A₃, A₄, R¹¹, B₁, B₂, B₄, B₅, R⁸, Q and W are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N.

Further suitable are compounds of the formula (Ia″), where the compounds of the formula (Ia″) are compounds of the formula (I-T22)

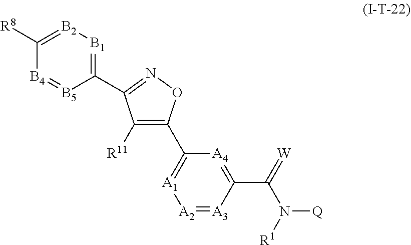

in which R¹, A₁, A₂, A₃, A₄, R¹¹, B₁, B₂, B₄, B₅, R⁸, Q and W are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N.

Further suitable are compounds of the formula (Ia″), where the compounds of the formula (Ia″) are compounds of the formula (I-T23)

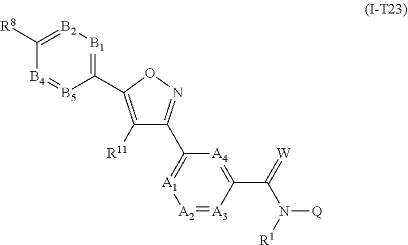

in which R¹, A₁, A₂, A₃, A₄, R¹¹, B₁, B₂, B₄, B₅, R⁸, Q and W are each defined as described herein, where not more than one moiety selected from A₁, A₂, A₃, A₄ is N and not more than one moiety selected from B₁, B₂, B₃, B₄ and B₅ is N; or where one or two moieties selected from A₁, A₂, A₃, A₄ may be N and not more than one moiety selected from B₁, B₂, B₃, B₄ and B₅ is N.

Further suitable are compounds according to the formulae and embodiments described herein, where R¹¹ is H and W is O.

Further suitable are compounds according to the formulae and embodiments described herein, where R¹¹ is H and W is 0 and B₃ is C—R⁸, R⁸ is halogen-substituted C₁-C₃-alkyl (preferably perhalogenated C₁-C₃-alkyl, more preferably perfluorinated C₁-C₃-alkyl) or halogen-substituted C₁-C₃-alkoxy (preferably perhalogenated C₁-C₃-alkoxy, more preferably perfluorinated C₁-C₃-alkoxy).

Further suitable are compounds according to the formulae and embodiments described herein, where the A₁ to A₄ and B₁ to B₅ moieties are as follows:
A₁ is C—H,
A₂ is CR³ or N,
A₃ is CR⁴,
A₄ is C—H,
B₁ is CR⁶ or N,
B₂ is C—H,
B₃ is CR⁸,
B₄ is C—H and
B₅ is CR¹⁰ or N.

Further suitable are compounds according to the formulae and embodiments described herein, where R¹ is H.

Further suitable are compounds according to the formulae and embodiments described herein, where Q is fluorine-substituted C₁-C₄-alkyl, C₃-C₄-cycloalkyl, optionally cyano- or fluorine-substituted C₃-C₄-cycloalkyl, C₄-C₆-heterocycloalkyl, 1-oxidothietan-3-yl, 1,1-dioxidothietan-3-yl, benzyl, pyridin-2-ylmethyl, methylsulfonyl or 2-oxo-2-(2,2,2-trifluoroethylamino)ethyl.

Further suitable are compounds according to the formulae and embodiments described herein, where R⁸ represents halogen or halogen-substituted C₁-C₄-alkyl.

Further suitable are compounds according to the formulae described herein, where R¹¹ is H.

Further suitable are compounds according to the formulae described herein, where R⁶, R⁷, R⁹ and R¹⁰ independently of one another are H, halogen, cyano, nitro, in each case optionally substituted C₁-C₄-alkyl, C₃-C₄-cycloalkyl, C₁-C₄-alkoxy, N-alkoxyiminoalkyl, C₁-C₄-alkylsulfanyl, C₁-C₄-alkylsulfinyl, C₁-C₄-alkylsulfonyl, N—C₁-C₄-alkylamino, N,N-di-C₁-C₄-alkylamino.

Further suitable are compounds according to the formulae described herein, where R², R³, R⁴ and R⁵ independently of one another are H, halogen, cyano, nitro, in each case optionally substituted C₁-C₄-alkyl, C₃-C₄-cycloalkyl, C₁-C₄-alkoxy, N—C₁-C₄-alkoxyimino-C₁-C₄-alkyl, C₁-C₄-alkylsulfanyl, C₁-C₄-alkylsulfinyl, C₁-C₄-alkylsulfonyl, N—C₁-C₄-alkylamino or N,N-di-C₁-C₄-alkylamino.

Further suitable are compounds according to the formulae described herein, where the A₁ to A₄ and B₁ to B₅ moieties are as follows:
A₁ is C—H,
A₂ is CR³ or N,
A₃ is CR⁴,
A₄ is C—H,
B₁ is CR⁶ or N,
B₂ is C—H,
B₃ is CR⁸,
B₄ is C—H and
B₅ is CR¹⁰ or N.

Further suitable are compounds according to the formulae described herein, where R¹ is H.

Further suitable are compounds according to the formulae described herein, where R¹ is methyl.

Further suitable are compounds according to the formulae described herein, where Q is fluorine-substituted or carbonamide (—C(═O)N(R)₂, where R independently of one another is H, C₁-C₃-alkyl or halogen-substituted C₁-C₃-alkyl)-substituted C₁-C₄-alkyl, optionally cyano- or fluorine-substituted C₃-C₄-cycloalkyl, C₄-C₆-heterocycloalkyl, 1-oxidothietan-3-yl, 1,1-dioxidothietan-3-yl, benzyl, pyridin-2-ylmethyl, methylsulfonyl or 2-oxo-2-(2,2,2-trifluoroethylamino)ethyl.

Further suitable are compounds according to the formulae described herein, where Q is 2,2,2-trifluoroethyl, 2,2-difluoroethyl, 3,3,3-trifluoropropyl, cyclopropyl, cyclobutyl, 1-cyanocyclopropyl, trans-2-fluorocyclopropyl, or cis-2-fluorocyclopropyl, oxetan-3-yl, thietan-3-yl, 1-oxidothietan-3-yl, 1,1-dioxidothietan-3-yl, benzyl, pyridin-2-ylmethyl, methylsulfonyl or 2-oxo-2-(2,2,2-trifluoroethylamino)ethyl.

Further suitable are compounds according to the formulae described herein, where R⁸ is halogen or halogen-substituted C₁-C₄-alkyl.

The following examples may be mentioned, which are preferred as active ingredients with parasiticidal effect for non-human organisms:

### 2-chloro-N-cyclopropyl-5-[1-[2,6-dichloro-4-[1,2,2,2-tetrafluoro-1-(trifluoromethyl)-ethyl]phenyl]pyrazol-4-yl]-N-methylpyridine-3-carboxamide

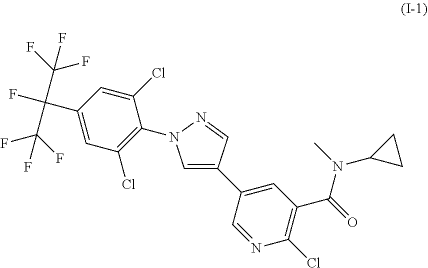 [SMILES: CN(C(=O)c1cc(-c2cnn(-c3c(Cl)cc(C(F)(C(F)(F)F)C(F)(F)F)cc3Cl)c2)cnc1Cl)C1CC1]

### 2-chloro-N-cyclopropyl-5-[1-[2-chloro-4-[1,2,2,2-tetrafluoro-1-(trifluoromethyl)ethyl](trifluoromethoxy)phenyl]-1H-pyrazol-4-yl]-3-pyridinecarboxamide

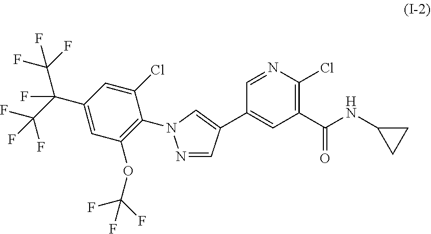 [SMILES: O=C(NC1CC1)c1cc(-c2cnn(-c3c(Cl)cc(C(F)(C(F)(F)F)C(F)(F)F)cc3OC(F)(F)F)c2)cnc1Cl]

### 2-chloro-5-[1-[2-chloro-4-[1,2,2,2-tetrafluoro-1-(trifluoromethyl)ethyl]-6-(trifluoromethyl)phenyl]-1H-pyrazol-4-yl]-N-cyclopropyl-3-pyridinecarboxamide

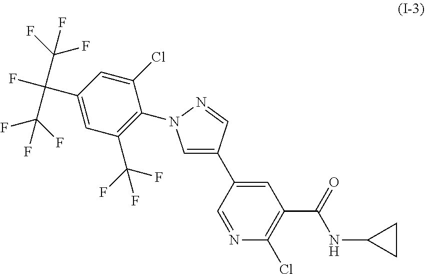 [SMILES: O=C(NC1CC1)c1cc(-c2cnn(-c3c(Cl)cc(C(F)(C(F)(F)F)C(F)(F)F)cc3C(F)(F)F)c2)cnc1Cl]

### 2-chloro-5-[1-[2-bromo-4-[1,2,2,2-tetrafluoro-1-(trifluoromethyl)ethyl](trifluoromethoxy)phenyl]-1H-pyrazol-4-yl]-N-cyclopropylpyridine-3-carboxamide

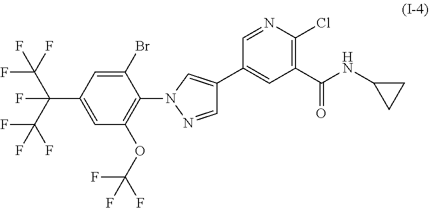 [SMILES: O=C(NC1CC1)c1cc(-c2cnn(-c3c(Br)cc(C(F)(C(F)(F)F)C(F)(F)F)cc3OC(F)(F)F)c2)cnc1Cl]

### 2-chloro-N-cyclopropyl-5-[1-[2,6-dichloro-4-[1,2,2,2-tetrafluoro (trifluoromethyl)ethyl]phenyl]pyrazol-4-yl]pyridine-3-carboxamide

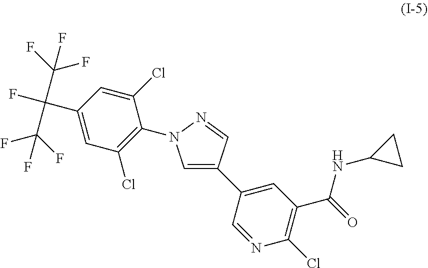 [SMILES: O=C(NC1CC1)c1cc(-c2cnn(-c3c(Cl)cc(C(F)(C(F)(F)F)C(F)(F)F)cc3Cl)c2)cnc1Cl]

### 2-chloro-N-cyanocyclopropyl-5-[1-[2-chloro-4-[1,2,2,2-tetrafluoro-1-(trifluoromethyl)ethyl]-6-(trifluoromethoxy)phenyl]-1H-pyrazol-4-yl]benzamide

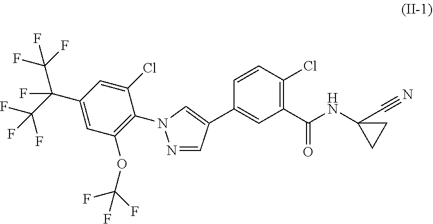 [SMILES: N#CC1(NC(=O)c2cc(-c3cnn(-c4c(Cl)cc(C(F)(C(F)(F)F)C(F)(F)F)cc4OC(F)(F)F)c3)ccc2Cl)CC1]

### 2-chloro-5-[1-[2-chloro-4-[1,2,2,2-tetrafluoro-1-(trifluoromethyl)ethyl]-6-(trifluoromethoxy)phenyl]pyrazol-4-yl]-N-cyclopropylbenzamide

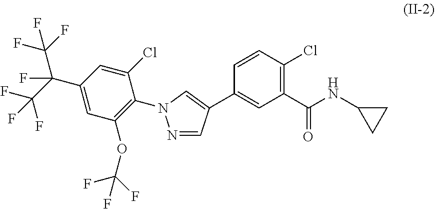 [SMILES: O=C(NC1CC1)c1cc(-c2cnn(-c3c(Cl)cc(C(F)(C(F)(F)F)C(F)(F)F)cc3OC(F)(F)F)c2)ccc1Cl]

### 5-[1-[2-bromo-4-[1,2,2,2-tetrafluoro-1-(trifluoromethyl)ethyl]-6-(trifluoromethoxy)phenyl]pyrazol-4-yl]-2-chloro-N-cyclopropylbenzamide

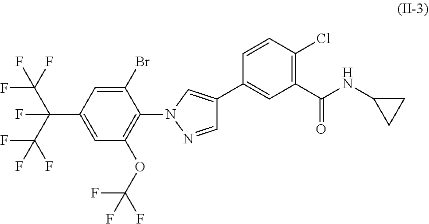 [SMILES: O=C(NC1CC1)c1cc(-c2cnn(-c3c(Br)cc(C(F)(C(F)(F)F)C(F)(F)F)cc3OC(F)(F)F)c2)ccc1Cl]

### 2-chloro-5-[1-[2-chloro-4-[1,2,2,2-tetrafluoro-1-(trifluoromethyl)ethyl](trifluoromethyl)phenyl]pyrazol-4-yl]-N-cyclopropylbenzamide

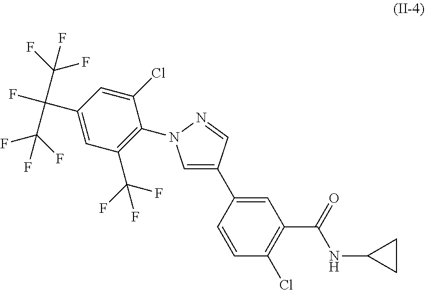 [SMILES: O=C(NC1CC1)c1cc(-c2cnn(-c3c(Cl)cc(C(F)(C(F)(F)F)C(F)(F)F)cc3C(F)(F)F)c2)ccc1Cl]

### 2-chloro-N-cyclopropyl-5-[1-[2,6-dichloro-4-[1,2,2,2-tetrafluoro-1-(trifluoromethyl)ethyl]phenyl]pyrazol-4-yl]benzamide

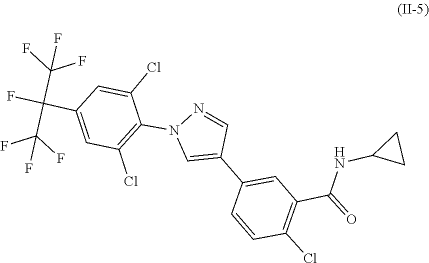 [SMILES: O=C(NC1CC1)c1cc(-c2cnn(-c3c(Cl)cc(C(F)(C(F)(F)F)C(F)(F)F)cc3Cl)c2)ccc1Cl]

### 2-chloro-N-cyclopropyl-5-[1-[2,6-dichloro-4-[1,2,2,2-tetrafluoro-1-(trifluoromethyl)ethyl]phenyl]pyrazol-4-yl]-N-methylbenzamide

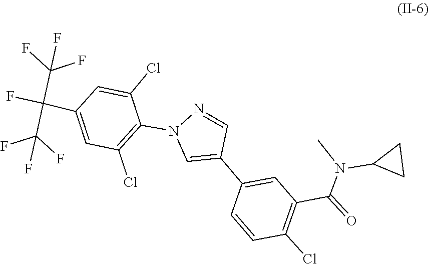 [SMILES: CN(C(=O)c1cc(-c2cnn(-c3c(Cl)cc(C(F)(C(F)(F)F)C(F)(F)F)cc3Cl)c2)ccc1Cl)C1CC1]

### 2-chloro-N-cyclopropyl-5-[1-[4-(1,1,1,2,3,3,3-heptafluoropropan-2-yl)-2-methyl-6-(trifluoromethyl)phenyl]-1H-pyrazol-4-yl]benzamide

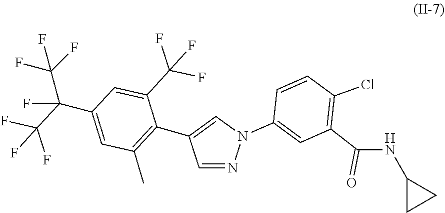 [SMILES: Cc1cc(C(F)(C(F)(F)F)C(F)(F)F)cc(C(F)(F)F)c1-c1cnn(-c2ccc(Cl)c(C(=O)NC3CC3)c2)c1]

### 2-chloro-N-(1-cyanocyclopropyl)-5-[1-[4-(1, 1,1,2,3,3,3-heptafluoropropan-2-yl)-2-methyl-6-(trifluoromethyl)phenyl]-1H-pyrazol-4-yl]benzamide

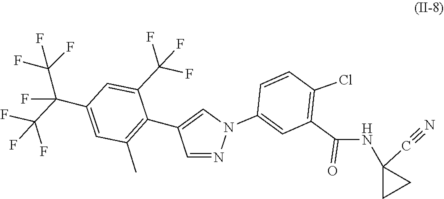 [SMILES: Cc1cc(C(F)(C(F)(F)F)C(F)(F)F)cc(C(F)(F)F)c1-c1cnn(-c2ccc(Cl)c(C(=O)NC3(C#N)CC3)c2)c1]

### 2-chloro-N-cyclopropyl-5-[4-[2,6-dimethyl-4-[1,2,2,2-tetrafluoro-1-(trifluoromethyl)ethyl]phenyl]pyrazol-1-yl]benzamide

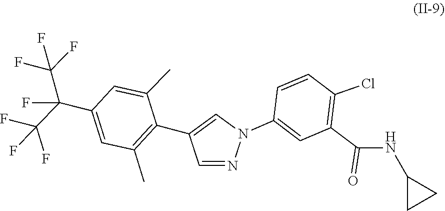 [SMILES: Cc1cc(C(F)(C(F)(F)F)C(F)(F)F)cc(C)c1-c1cnn(-c2ccc(Cl)c(C(=O)NC3CC3)c2)c1]

### 2-chloro-N-(1-cyanocyclopropyl)-5-[4-[2,6-dichloro-4-[1,2,2,2-tetrafluoro-1-(trifluoromethyl)ethyl]phenyl]pyrazol-1-yl]benzamide

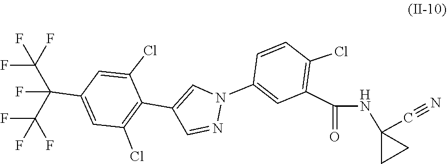 [SMILES: N#CC1(NC(=O)c2cc(-n3cc(-c4c(Cl)cc(C(F)(C(F)(F)F)C(F)(F)F)cc4Cl)cn3)ccc2Cl)CC1]

### 2-chloro-5-[3-[2-chloro-4-[1,2,2,2-tetrafluoro-1-(trifluoromethyl)ethyl]-6-(trifluoromethyl)phenyl]isoxazol-5-yl]-N-cyclopropylbenzamide

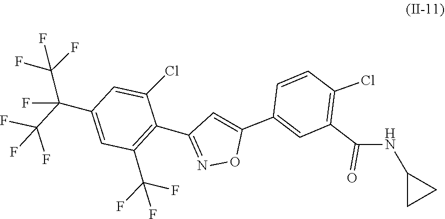 [SMILES: O=C(NC1CC1)c1cc(-c2cc(-c3c(Cl)cc(C(F)(C(F)(F)F)C(F)(F)F)cc3C(F)(F)F)no2)ccc1Cl]

### 2-chloro-N-(1-cyanocyclopropyl)-5-[3-[2-methyl-4- [ 1,2,2,2-tetrafluoro (trifluoromethyl)ethyl]-6-(trifluoromethyl)phenyl]isoxazol-5-yl]benzamide

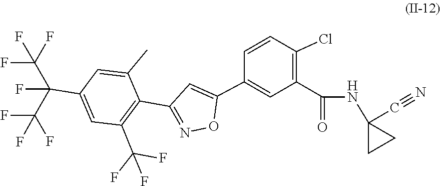 [SMILES: Cc1cc(C(F)(C(F)(F)F)C(F)(F)F)cc(C(F)(F)F)c1-c1cc(-c2ccc(Cl)c(C(=O)NC3(C#N)CC3)c2)on1]

### 2-chloro-N-(1-cyanocyclopropyl)-5-[1-[2,6-dichloro-4-[1,2,2,2-tetrafluoro-1-(trifluoromethyl)ethyl]phenyl]pyrrol-3-yl]benzamide

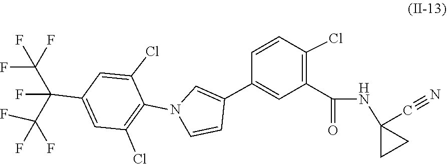 [SMILES: N#CC1(NC(=O)c2cc(-c3ccn(-c4c(Cl)cc(C(F)(C(F)(F)F)C(F)(F)F)cc4Cl)c3)ccc2Cl)CC1]

### 2-chloro-5-[3-[2-chloro-4-[1,2,2,2-tetrafluoro-1-(trifluoromethyl)ethyl]-6-(trifluoromethyl)phenyl]pyrrol-1-yl]-N-cyclopropylbenzamide

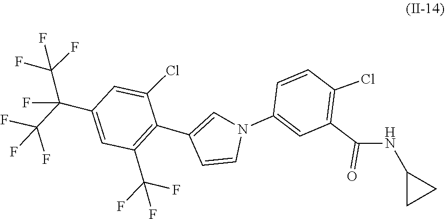 [SMILES: O=C(NC1CC1)c1cc(-n2ccc(-c3c(Cl)cc(C(F)(C(F)(F)F)C(F)(F)F)cc3C(F)(F)F)c2)ccc1Cl]

The active ingredients described above may in this case be part of the formulation described herein and thus enable the efficacy against parasites described in detail as follows.

In the context of the present invention, preference may be given to the active ingredients of the formulae I-1 to I-5 and the active ingredients of the formulae II-1 to II-6 described above, wherein particular preference may be given to active ingredients according to the formulae I-1 to I-5.

According to a preferred embodiment, the compound according to the formula I-1 can be present as active ingredient, in which a mixture of isopropanol (32.5), ethanol (32.5) and propylene carbonate (35) can be used as solvent mixture or a mixture of isopropanol (32.5), ethanol (32.5) and glycerol carbonate (35), or a mixture of isopropanol (65), propylene carbonate (17.5) and glycerol carbonate (17.5) or also a mixture of ethanol (65), propylene carbonate (17.5) and glycerol carbonate (17.5), where the numbers in brackets are intended to represent the content in % by weight, based on the solvent mixture.

According to a further embodiment, the solvent mixtures specified above can be used for an active ingredient according to the formula I-2, or the formula I-3 or the formula I-4 or the formula I-5.

However, preference is given in particular to the active ingredients according to the formulae I-1, 1-2,1-3, 1-4 and I-5 using only the first and the second solvent.

In addition to the constituents listed above, the composition according to the invention may also comprise customary pharmaceutically acceptable auxiliaries. Examples of these which may be mentioned are: spreaders, antioxidants, pH regulators, crystallization inhibitors, surfactants, dyes, pigments, penetration enhancers and preservatives.

Spreaders are, for example, spreading oils such as di-2-ethylhexyl adipate, isopropyl myristate, dipropylene glycol pelargonate, cyclic and acyclic silicone oils such as dimethicones and also copolymers and terpolymers thereof with ethylene oxide, propylene oxide and formalin, fatty acid esters, triglycerides, fatty alcohols. They can be added additionally at a proportion of ≥1−≤40% w/v. According to additional embodiments spreaders may be present at proportions of ≥0.01-≤1% w/v, ≥0.1-≤10% w/v, ≥1-≤20% w/v, ≥20-≤50% w/v or ≥10-≤30% w/v.

Antioxidants are, for example, ascorbic acid, di-Na-EDTA, BHT, BHA, delta-tocopherol, thioglycerol. They can be added additionally at a proportion of ≥0.01−≤2% w/v.

According to additional embodiments antioxidants may be present at proportions of ≥0.01−≤1% w/v, ≥0.05−≤0.5% w/v or ≥1−≤2% w/v.

pH regulators are preferably organic acids or bases, for example citric acid, triethanolamine. They can be added additionally at a proportion of ≥0.001−≤5% w/v.

According to additional embodiments pH regulators may be present at proportions of ≥0.01−≤1% w/v, ≥0.05−≤0.5% w/v or ≥0.1−≤3% w/v.

Crystallization inhibitors are, for example, cellulose ethers such as hydroxypropylmethylcellulose (HPMC); polyvinylpyrrolidone (PVP). They can be added additionally at a proportion of ≥0.1−≤50% w/v. According to additional embodiments crystallization inhibitors may be present at proportions of ≥1−≤30% w/v, ≥1−≤20% w/v, ≥5−≤15% w/v, ≥1−≤20% w/v or ≥0.1−≤10% w/v.

Surfactants which may be mentioned are: non-ionic surfactants, e.g. polyoxyethylated castor oil, polyoxyethylated sorbitan monooleate, sorbitan monostearate, glycerol monostearate, polyoxyethyl stearate, alkylphenol polyglycol ether; ampholytic surfactants such as di-Na—N-lauryl β-iminodipropionate or lecithin; anion-active surfactants such as Na-lauryl sulfate, fatty alcohol ether sulfates, mono/dialkyl polyglycol ether orthophosphoric acid ester monoethanolamine salt; cation-active surfactants such as cetyltrimethylammonium chloride. They can be added additionally at a proportion of ≥0.1−≤20% w/v. According to further embodiments they may be present at proportions of ≥0.1−≤10% w/v, ≥0.1−≤1% w/v, ≥1−≤10% w/v or ≥0.2−≤5% w/v.

Dyes can also be added to the pharmaceutical composition. They may be dissolved in the composition or suspended. Examples which may be mentioned are Brilliant Blue, D&C Violet No. 2, FD&C Yellow, Sunset Yellow FCF or natural food colorings such as chlorophyllin. They can be added additionally at a proportion of ≥0.01−≤2% w/v. According to additional embodiments they may be present at a proportion of ≥0.01−≤1% w/v or ≥0.1−≤2% w/v.

The pharmaceutical compositions may contain penetration enhancers such as: menthol, 1,8-cineol, glycol ethers (e.g. diethylene glycol methyl ether, diethylene glycol monoethyl ether, dipropylene glycol monoethyl ether, diethylene glycol monobutyl ether). They may be present at a proportion of ≥2−≤50% w/v. According to additional embodiments penetration enhancers may be present at proportions of ≥1−≤20% w/v, ≥0.5−≤20% w/v, ≥0.1−≤10% w/v, ≥10−≤50% w/v or ≥10−≤40% w/v.

In principle, the concentration of the active ingredient is unrestricted, but consideration should also be given to the fact that all active ingredient should be in solution. With regard to an advantageous pour-on application, for high efficacy, it can also be preferable for the pharmaceutically active ingredient to be present in an amount of greater than or equal to 0.1% by weight to less than or equal to 8% by weight, for example greater than or equal to 0.1% by weight to less than or equal to 5% by weight. This can correspond, for example, to a proportion of 0.2-4 g/100 ml, for example 1-3 g/100 ml, based on the pharmaceutical composition. Such concentrations can be realized without difficulty by the configuration of the solvent mixture described above and are particularly advantageous for a topical pour-on application. In principle, however, also concentrations of the active ingredient of up to 30% by weight or above are possible without going beyond the scope of the invention.

The formulations according to the invention are therefore typically liquid and are suitable for topical or dermal application, especially as so-called pour-on formulations.

The ectoparasiticidal efficacy of the compositions according to the invention comprising active ingredients from the aforementioned group of in particular fluorinated benzamides is very effectively provided, especially in the case of the pour-on application described above. By use of this composition, therefore, the amount of active ingredient required can be reduced and the long-term effect can be increased. Accordingly, their use achieves economic and ecological advantages.

The compositions according to the invention are outstandingly suitable for use in parasite control or parasite prophylaxis. Thus, the pharmaceutical compositions described here serve for use in the treatment of or prophylaxis of parasite infestation, especially in the treatment of or prophylaxis of ectoparasites.

The compositions according to the invention are particularly suitable in the control of or prophylaxis of ectoparasites, preferably of ticks and/or mites, and/or flies or fly larvae, and/or lice, on non-human organisms, especially animals, particularly warm-blooded animals, preferably mammals.

According to one embodiment animals may be domestic animals for example cage birds; or mammals such as hamsters, guinea pigs, rats, mice, chinchillas, ferrets or in particular dogs, cats.

According to a further, more preferred embodiment animals may be agricultural livestock which includes, for example, poultry, such as turkeys, ducks, geese, and in particular chickens; or preferably mammals, such as, sheep, goats, horses, donkeys, camels, buffaloes, rabbits, reindeers, fallow deers, and in particular cattle and pigs.

According to a particularly preferred embodiment the present compositions are used in the control of and/or prophylaxis of ectoparasites on cattle.

Since the treated animals also generally distribute a certain amount of the composition used into the environment, e.g. by abrasion or with debris, the effect of the composition according to the invention possibly occurs not only directly on the animal but also to a corresponding extent in their environment.

As described above, the composition described here is suitable preferably in the treatment or prophylaxis of parasite infestation in non-human organisms by dripping or pouring the composition onto the non-human organism, and thus for a pour-on application.

Parasites that may be mentioned against which the formulations of the invention may be effective are:

from the order of Anoplura, e.g. Haematopinus spp., Linognathus spp., Solenopotes spp., Pediculus spp., Pthirus spp.;

from the order of Mallophaga, e.g. Trimenopon spp., Menopon spp., Eomenacanthus spp., Menacanthus spp., Trichodectes spp., Felicola spp., Damalinea spp., Bovicola spp; from the order of Diptera z.B. Aedes spp., Anopheles spp., Culex spp., Simulium spp., Phlebotomus spp., Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfartia spp., Gasterophilus spp., Oesteromyia spp., Oedemagena spp., Hypoderma spp., Oestrus spp., Rhinoestrus spp., Melophagus spp., Hippobosca spp..

from the order of Siphonaptera, e.g. Ctenocephalides spp., Echidnophaga spp., Ceratophyllus spp., Pulex spp.

from the order of Metastigmata, e.g. Hyalomma spp., Rhipicephalus spp. (including the genus formerly referred to as Boophilus), Amblyomma spp., Haemaphysalis spp., Dermacentor spp., Ixodes spp., Argas spp., Ornithodorus spp., Otobius spp.;

from the order of Mesostigmata, e.g. Dermanyssus spp., Ornithonyssus spp., Pneumonyssus spp..

from the order of Prostigmata, e.g. Cheyletiella spp., Psorergates spp., Myobia spp., Demodex spp., Neotrombi-cula spp.;

from the order of Astigmata, e.g. Acarus spp., Myocoptes spp., Psoroptes spp., Chorioptes spp., Otodectes spp., Sarcoptes spp., Notoedres spp., Knemidocoptes spp., Neoknemidocoptes spp. Cytodites spp., Laminosioptes spp..

Here, from the order of the Diptera are particularly preferred Tabanus spp., Musca spp., Hydrotaea spp., Haematobia spp., Stomoxys spp., Glossina spp., Lucilia spp., Calliphora spp., Aedes spp., Anopheles spp., Culex spp., Simulium spp., Phlebotomus spp.; from the order of Metastigmata Hyalomma spp., Rhipicephalus spp. (including the genus formerly referred to as Boophilus), Amblyomma spp., Haemaphysalis spp., Dermacentor spp., Ixodes spp., Argas spp., Ornithodorus spp., Otobius spp.; from the order of Anoplura Haematopinus spp., Linognathus spp., Solenopotes spp.;

from the order of Mallophaga Damalinea spp., Bovicola spp., Trichodectes spp.; from the order of Mesostigmata Dermanyssus spp., Ornithonyssus spp.; from the order of Astigmata Psoroptes spp., Chorioptes spp., Otodectes spp., Sarcoptes spp., Notoedres spp..

To be mentioned as particularly preferred are arthropods of the species or genera Haematobia irritans irritans, Haematobia irritans exigua, Hydrotea irritans, Musca autumnalis, Culicoides spp., Stomoxys calcitrans, Glossina spp., Aedes spp., Anopheles spp., Culex spp., Damalinia (Bovicola) bovis, Linognathus vituli, Haematopinus eurystemus, Solenopotes capillatus, Psoroptes bovis, Rhipicephalus (Boophilus) microplus, Rhipicephalus (Boophilus) annulatus, Rhipicephalus (Boophilus) decoloratus, Ixodes ricinus, Amblyomma maculatum, Amblyomma variegatum, Ixodes scapularis, Ixodes holocyclus, Haemaphysalis longicomis, Otobius megnini.

The compositions according to the invention can be prepared by customary methods, for example by mixing the active ingredients with the further constituents with stirring and producing a solution. This can optionally be filtered. Suitable for filling are plastic tubes for example.

The liquid formulations according to the invention are characterized by their excellent storage stability of at least one year, preferably at least two years, more preferably at least three years in all climate zones. Owing to the very high efficacy, the application volumes can be kept low. The preferred application volumes are 0.01-1 ml/kg [bodyweight of the animal to be treated], preferably 0.05-0.1 ml/kg [bodyweight of the animal to be treated].

The compositions according to the invention also have excellent skin compatibility and have low toxicity. Finally, they are environmentally friendly due to their biodegradability.

Solubility Experiments

Dissolution experiments for active ingredients I-1, I-2, I-3 and I-4 in solvents for a pharmaceutical composition according to the invention are presented below. For biological effect, the active ingredient should be incorporated at a certain dose or amount in the formulation, in the ideal case it should be a solution in the formulation such that the active ingredient is therefore dissolved in the solvent. A certain minimum solubility is required for the solvent since the application volume is limited.

Table 1 shows the results of the dissolution experiments for active ingredient I-1. In this case, 10 g of the appropriate solvent or solvent mixture were initially charged and as much active ingredient was added until the solution is saturated. Isopropanol (IPA) and propylene carbonate (PPC) were used as solvents. The samples were stirred for 3 days. Should the active ingredient become fully dissolved, active ingredient was again added. Subsequently, the samples were stored for a further 4 days at the relevant temperature and centrifuged in order to remove the undissolved particles. The supernatants were analyzed in each case for active ingredient content by HPLC-UV.

TABLE 1

| Solvent (mixture) | Saturation |
|---|---|
| and temperature | concentration |
| of the solution | %[w/w] |
| Isopropanol 5° C. | 0.98 |
| Isopropanol 23° C. | 1.56 |
| Propylene carbonate 5° C. | 1.80 |
| Propylene carbonate 23° C. | 2.43 |
| IPA/PPC 25/75 23° C. | 7.58 |
| IPA/PPC 35/65 23° C. | 8.79 |
| IPA/PPC 50/50 23° C. | 9.26 |
| IPA/PPC 60/40 23° C. | 8.60 |
| IPA/PPC 65/35 5° C. | 6.32 |
| IPA/PPC 65/35 23° C. | 8.31 |
| IPA/PPC 70/30 23° C. | 7.49 |
| IPA/PPC 75/25 23° C. | 6.70 |
| IPA/PPC 80/20 23° C. | 5.90 |
| IPA/PPC 90/10 23° C. | 3.74 |

It can be seen from Table 1 that in the singular solvents isopropanol and propylene carbonate, the desired criteria have not been met, particularly with regard to sufficient solubility. Particularly preferred desired solubilities of the active ingredient that are advantageous and in accordance with the invention are in a range of at least 5% m/V, in which sufficient gap from the saturation solubility should be present. Unexpectedly, a significant solubility increase in the mixture of isopropanol and propylene carbonate compared to the pure solvents was shown, which is presented in detail in Table 1.

Furthermore, solubility experiments with three further active ingredients from the class of fluorinated heteroarylamides in different solvents were carried out. The solubility experiments were carried out analogously to the experiments described above, however only at room temperature and the solvent mixture isopropanol/propylene carbonate 65:35% [m/m] was selected. The results are listed in Table 2.

TABLE 2

| | | Saturation |
|---|---|---|
| Active | | solubility |
| compound | Solvent | % [w/w] |
| I-2 | Isopropanol | 6.99 |
| I-2 | Propylene carbonate | 5.34 |
| I-2 | Isopropanol/propylene carbonate 65/35 | 24.20 |
| I-3 | Isopropanol | 4.18 |
| I-3 | Propylene carbonate | 4.89 |
| I-3 | Isopropanol/propylene carbonate 65/35 | 17.57 |
| I-4 | Isopropanol | 13.42 |
| I-4 | Propylene carbonate | 11.76 |
| I-4 | Isopropanol/propylene carbonate 65/35 | 29.38 |

It was also found for active ingredients I-2, I-3 and I-4 that very good solubilities for the active ingredient of the family of heteroarylamides, especially for the defined structures, could be achieved by the solvent mixture or solvent phase of the composition according to the invention.

It was also shown that the significant solubility increase was not limited to mixtures of isopropanol and propylene carbonate, but was also shown for other solvents of the series of alcohols and carbonates, as defined above for the first and the second solvent.

TABLE 3

| Active | | Saturation solubility |
|---|---|---|
| compound | Solvent | % [ w/w] |
| I-1 | Isopropanol | 1.56 |
| I-1 | Propylene carbonate | 2.43 |
| I-1 | Ethanol | 2.76 |
| I-1 | Glycerol carbonate | 0.15 |
| I-1 | Isopropanol/glycerol | 5.98 |
| | carbonate 65:35 | |
| I-1 | Ethanol/glycerol carbonate | 6.48 |
| | 65:35 | |
| I-1 | Ethanol/propylene | 9.45 |
| | carbonate 65:35 | |

It can clearly be seen in Table 3 that the increased solubilities could be achieved with other solvents. Therefore, the invention can extend to other solvents within the range described. Shown as examples here are the experiments with ethanol and glycerol carbonate, the mixtures of which and combinations of which with the solvents already shown, isopropanol and propylene carbonate.

Experiments with solvent systems were also carried out in which the first solvent comprised two alcohols. The results are shown in Table 4.

TABLE 4

| Active | | Solubility % |
|---|---|---|
| compound | Solvent | [w/w] |
| I-1 | Isopropanol/ethanol/propylene | >5 |
| | carbonate 32.5:32.5:35 | |
| I-1 | Isopropanol/ethanol/glycerol | >5 |
| | carbonate 32.5:32.5:35 | |
| I-1 | Isopropanol/propylene | >5 |
| | carbonate/glycerol carbonate | |
| | 65:17.5:17.5 | |
| I-1 | Ethanol/propylene | >5 |
| | carbonate/glycerol carbonate | |
| | 65:17.5:17.5 | |

It could also be shown that such binary systems enable good solubilities for the active ingredient according to the formula I-1.

### FORMULATION EXAMPLES

Described below are preparation examples for formulations with active ingredients I-1, I-2, I-3 and I-4 as defined above with additives. In all examples specified as carried out, the respective active ingredient was fully dissolved and the stability was as desired.

Here, Table 5 shows appropriate formulation examples with data of the solvent mixture, active ingredient and optionally further additives.

TABLE 5

| No. | Constituents | Amount | |
|---|---|---|---|
| 1 | I-1 | 1.5 | g |
| | Butylhydroxytoluene | 0.1 | g |
| | Isopropanol/propylene carbonate 65:35% w/w | to 100 | mL |
| 2 | I-1 | 2 | g |
| | Butylhydroxytoluene | 0.1 | g |
| | Isopropanol/propylene carbonate 65:35% w/w | to 100 | mL |
| 3 | I-1 | 4.5 | g |
| | Butylhydroxytoluene | 0.1 | g |
| | Isopropanol/propylene carbonate 65:35% w/w | to 100 | mL |
| 4 | I-1 | 4.5 | g |
| | Isopropanol/propylene carbonate 65:35% w/w | to 100 | mL |
| 5 | I-1 | 0.5 | g |
| | Isopropanol/propylene carbonate 65:35% w/w | to 100 | mL |
| 6 | I-4 | 4.5 | g |
| | water | 1 | g |
| | Isopropanol/propylene carbonate 65:35% w/w | to 100 | mL |
| 7 | I-1 | 1 | g |
| | Isopropanol/propylene carbonate 65:35% w/w | to 100 | mL |
| 8 | I-1 | 2 | g |
| | Isopropanol/propylene carbonate 65:35% w/w | to 100 | mL |
| 9 | I-1 | 4 | g |
| | Isopropanol/propylene carbonate 65:35% w/w | to 100 | mL |
| 10 | I-1 | 0.25 | g |
| | Isopropanol/propylene carbonate 65:35% w/w | to 100 | mL |
| 11 | I-4 | 3 | g |
| | Butylhydroxytoluene | 0.1 | g |
| | Isopropanol/propylene carbonate 65:35% w/w | to 100 | mL |
| 12 | I-4 | 2 | g |
| | Butylhydroxytoluene | 0.1 | g |
| | Isopropanol/propylene carbonate 65:35% w/w | to 100 | mL |
| 13 | I-2 | 3 | g |
| | Butylhydroxytoluene | 0.1 | g |
| | Isopropanol/propylene carbonate 65:35% w/w | to 100 | mL |
| 14 | I-2 | 3 | g |
| | Isopropanol/propylene carbonate 65:35% w/w | to 100 | mL |
| 15 | I-3 | 1.5 | g |
| | Butylhydroxytoluene | 0.1 | g |
| | Isopropanol/propylene carbonate 65:35% w/w | to 100 | mL |
| 16 | I-3 | 4.5 | g |
| | Butylhydroxytoluene | 0.1 | g |
| | Isopropanol/propylene carbonate 65:35% w/w | to 100 | mL |
| 17 | I-4 | 2 | g |
| | Isopropanol/propylene carbonate 65:35% w/w | to 100 | mL |
| 18 | I-1 | 1.9 | g |
| | Isopropanol/propylene carbonate 65:35% w/w | to 100 | mL |
| 19 | I-1 | 2 | g |
| | Ascorbic acid | 0.1 | g |
| | Isopropanol/propylene carbonate 65:35% w/w | to 100 | mL |
| 20 | I-4 | 4.5 | g |
| | Water | 2 | g |
| | Isopropanol/propylene carbonate 65:35% w/w | to 100 | mL |
| 21 | I-1 | 2 | g |
| | Ascorbic acid | 0.5 | g |
| | Isopropanol/propylene carbonate 65:35% w/w | to 100 | mL |
| 22 | I-1 | 2 | g |
| | Disodium EDTA | 0.09 | g |
| | Isopropanol/propylene carbonate 65:35% w/w | to 100 | mL |
| 23 | I-1 | 2 | g |
| | Brilliant Blue | 0.1 | g |
| | Isopropanol/propylene carbonate 65:35% w/w | to 100 | mL |
| 24 | I-1 | 2 | g |
| | Brilliant Blue | 0.0225 | g |
| | Isopropanol/propylene carbonate 65:35% w/w | to 100 | mL |
| 25 | I-1 | 2 | g |
| | Delta-Tocopherol | 0.1 | g |
| | Isopropanol/propylene carbonate 65:35% w/w | to 100 | mL |
| 26 | I-1 | 2 | g |
| | Thioglycerol | 0.5 | g |
| | Isopropanol/propylene carbonate 65:35% w/w | to 100 | mL |
| 27 | I-1 | 2 | g |
| | Butylhydroxytoluene | 0.1 | g |
| | Butylhydroxyanisole | 0.1 | g |
| | Citric acid | 0.3 | g |
| | Isopropanol/propylene carbonate 65:35% w/w | to 100 | mL |
| 28 | I-1 | 2 | g |
| | Citric acid | 0.3 | g |
| | Isopropanol/propylene carbonate 65:35% w/w | to 100 | g |
| 29 | I-1 | 2 | g |
| | Butylhydroxyanisole | 0.1 | g |
| | Isopropanol/propylene carbonate 65:35% w/w | to 100 | mL |
| 30 | I-1 | 2 | g |
| | D&C Violet No.2 | 0.0225 | g |
| | Isopropanol/propylene carbonate 65:35% w/w | to 100 | mL |
| 31 | I-1 | 2 | g |
| | Water | 0.2 | g |
| | Isopropanol/propylene carbonate 65:35% w/w | to 100 | mL |
| 32 | I-1 | 2 | g |
| | Water | 0.4 | g |
| | Isopropanol/propylene carbonate 65:35% w/w | to 100 | mL |
| 33 | I-1 | 2 | g |
| | Water | 2 | g |
| | Isopropanol/propylene carbonate 65:35% w/w | to 100 | mL |
| 34 | I-1 | 2 | g |
| | Water | 3 | g |
| | Isopropanol/propylene carbonate 65:35% w/w | to 100 | mL |
| 35 | I-1 | 2 | g |
| | Water | 5 | g |
| | Isopropanol/propylene carbonate 65:35% w/w | to 100 | mL |
| 36 | I-1 | 2 | g |
| | Water | 5 | g |
| | Triethanolamine | 0.06 | g |
| | Isopropanol/propylene carbonate 65:35% w/w | to 100 | mL |
| 37 | I-1 | 2 | g |
| | Water | 5 | g |
| | Citric acid | 0.00006 | g |
| | Isopropanol/propylene carbonate 65:35% w/w | to 100 | mL |
| 38 | I-1 | 1.99 | g |
| | Water | 10 | g |
| | Isopropanol/propylene carbonate 65:35% w/w | to 100 | mL |
| 39 | I-1 | 1.99 | g |
| | Water | 5 | g |
| | Isopropanol/propylene carbonate 61.3:31.5% w/w | to 100 | mL |
| 40 | I-1 | 1.99 | g |
| | Water | 0.5 | g |
| | Isopropanol/propylene carbonate 65:35% w/w | to 100 | mL |
| 41 | I-1 | 1.99 | g |
| | Water | 2 | g |
| | Isopropanol/propylene carbonate 65:35% w/w | to 100 | mL |
| 42 | I-1 | 1.99 | g |
| | Water | 1 | g |
| | Isopropanol/propylene carbonate 65:35% w/w | to 100 | mL |
| 43 | I-1 | 2.1 | g |
| | Isopropanol/propylene carbonate 65:35% w/w | to 100 | mL |
| 44 | I-1 | 2 | g |
| | Isopropanol/propylene carbonate 68:32% w/w | to 100 | mL |
| 45 | I-1 | 1.99 | g |
| | Isopropanol/propylene carbonate 65:35% w/w | to 100 | mL |
| 46 | I-4 | 4.5 | g |
| | Water | 5 | g |
| | Isopropanol/propylene carbonate 65:35% w/w | to 100 | mL |
| 47 | I-1 | 4.5 | g |
| | Water | 1 | g |
| | Isopropanol/propylene carbonate 65:35% w/w | to 100 | mL |
| 48 | I-1 | 4.5 | g |
| | Water | 2 | g |
| | Isopropanol/propylene carbonate 65:35% w/w | to 100 | mL |
| 49 | I-1 | 4.5 | g |
| | Water | 5 | g |
| | Isopropanol/propylene carbonate 65:35% w/w | to 100 | mL |
| 50 | I-2 | 2 | g |
| | Butylhydroxytoluene | 0.1 | g |
| | Isopropanol/propylene carbonate 65:35% w/w | to 100 | mL |
| 51 | I-1 | 2 | g |
| | Ethanol/propylene carbonate 65:35% w/w | to 100 | mL |
| 52 | I-4 | 4.5 | g |
| | Isopropanol/propylene carbonate 65:35% w/w | to 100 | mL |
| 53 | I-1 | 2 | g |
| | Isopropanol/propylene carbonate 62:38% w/w | to 100 | mL |
| 54 | I-1 | 35 | g |
| | N-methylpyrrolidone | to 100 | mL |
| 55 | I-1 | 2 | g |
| | Propylene carbonate | to 100 | mL |
| 56 | I-1 | 1 | g |
| | Isopropanol | to 100 | mL |
| 57 | I-2 | 2 | g |
| | Isopropanol/propylene carbonate 65:35% w/w | to 100 | mL |
| 58 | I-2 | 4.5 | g |
| | Isopropanol/propylene carbonate 65:35% w/w | to 100 | mL |
| 59 | I-1 | 4.5 | g |
| | Isopropanol/ethanol/propylene carbonate | to 100 | mL |
| | 32.5:32.5:35% w/w | | |
| 60 | I-1 | 4.5 | g |
| | Isopropanol/ethanol/glycerol carbonate | to 100 | mL |
| | 32.5:32.5:35% w/w | | |
| 61 | I-1 | 4.5 | g |
| | Isopropanol/propylene carbonate/glycerol | to 100 | mL |
| | carbonate 65:17.5:17.5% w/w | | |
| 62 | I-1 | 4.5 | g |
| | Ethanol/propylene carbonate/glycerol carbonate | to 100 | mL |
| | 65:17.5:17.5% w/w | | |
| 63 | I-2 | 4.5 | g |
| | Water | 1 | g |
| | Isopropanol/propylene carbonate 65:35% w/w | to 100 | mL |
| 64 | I-2 | 4.5 | g |
| | Water | 2 | g |
| | Isopropanol/propylene carbonate 65:35% w/w | to 100 | mL |
| 65 | I-2 | 4.5 | g |
| | Water | 5 | g |
| | Isopropanol/propylene carbonate 65:35% w/w | to 100 | mL |

Table 6 shows formulations which can be prepared according to the solubility experiments.

TABLE 6

| No. | Constituents | Amount | |
|---|---|---|---|
| 66 | I-1 | 1.5 | g |
| | Isopropanol/propylene carbonate 65:35% w/w | to 100 | mL |
| 67 | I-1 | 3 | g |
| | Ethanol/propylene carbonate 65:35% w/w | to 100 | mL |
| 68 | I-1 | 1.99 | g |
| | Ethanol/propylene carbonate 65:35% w/w | to 100 | mL |
| 69 | I-1 | 3 | g |
| | Ethanol/glycerol carbonate 65:35% w/w | to 100 | mL |
| 70 | I-1 | 1.99 | g |
| | Ethanol/glycerol carbonate 65:35% w/w | to 100 | mL |
| 71 | I-1 | 3 | g |
| | Isopropanol/glycerol carbonate 65:35% w/w | to 100 | mL |
| 72 | I-1 | 1.99 | g |
| | Isopropanol/glycerol carbonate 65:35% w/w | to 100 | mL |
| 73 | I-1 | 3 | g |
| | Benzyl alcohol/glycerol carbonate 65:35% w/w | to 100 | mL |
| 74 | I-1 | 1.99 | g |
| | Benzyl alcohol/glycerol carbonate 65:35% w/w | to 100 | mL |
| 75 | I-1 | 3 | g |
| | Benzyl alcohol/propylene carbonate 65:35% w/w | to 100 | mL |
| 76 | I-1 | 1.99 | g |
| | Benzyl alcohol/propylene carbonate 65:35% w/w | to 100 | mL |
| 77 | I-1 | 1.5 | g |
| | 1-Methoxy-2-propanol/glycerol carbonate | to 100 | mL |
| | 65:35% w/w | | |
| 78 | I-1 | 4.5 | g |
| | 1-Methoxy-2-propanol/propylene carbonate | to 100 | mL |
| | 65:35% w/w | | |
| 79 | I-1 | 1.5 | g |
| | 1-Methoxy-2-propanol/propylene carbonate | to 100 | mL |
| | 65:35% w/w | | |
| 80 | I-2 | 1.5 | g |
| | Isopropanol/propylene carbonate 65:35% w/w | to 100 | mL |
| 81 | I-3 | 4.5 | g |
| | Isopropanol/propylene carbonate 65:35% w/w | to 100 | mL |
| 82 | I-3 | 1.5 | g |
| | Isopropanol/propylene carbonate 65:35% w/w | to 100 | mL |
| 83 | I-4 | 4.5 | g |
| | Isopropanol/propylene carbonate 65:35% w/w | to 100 | mL |
| 84 | I-4 | 1.5 | g |
| | Isopropanol/propylene carbonate 65:35% w/w | to 100 | mL |
| 85 | I-1 | 3 | g |
| | Isopropanol/propylene carbonate 25:75% w/w | to 100 | mL |
| 86 | I-1 | 3 | g |
| | Isopropanol/propylene carbonate 65:35% w/w | to 100 | mL |
| 87 | I-1 | 4.5 | g |
| | Ethanol/propylene carbonate 65:35% w/w | to 100 | mL |
| 88 | I-1 | 1.5 | g |
| | Ethanol/propylene carbonate 65:35% w/w | to 100 | mL |
| 89 | I-1 | 4.5 | g |
| | Ethanol/glycerol carbonate 65:35% w/w | to 100 | mL |
| 90 | I-1 | 1.5 | g |
| | Ethanol/glycerol carbonate 65:35% w/w | to 100 | mL |
| 91 | I-1 | 4.5 | g |
| | Isopropanol/glycerol carbonate 65:35% w/w | to 100 | mL |
| 92 | I-1 | 1.5 | g |
| | Isopropanol/glycerol carbonate 65:35% w/w | to 100 | mL |
| 93 | I-1 | 4.5 | g |
| | Benzyl alcohol/glycerol carbonate 65:35% w/w | to 100 | mL |
| 94 | I-1 | 1.5 | g |
| | Benzyl alcohol/glycerol carbonate 65:35% w/w | to 100 | mL |
| 95 | I-1 | 4.5 | g |
| | Benzyl alcohol/propylene carbonate 65:35% w/w | to 100 | mL |
| 96 | I-1 | 1.5 | g |
| | Benzyl alcohol/propylene carbonate 65:35% w/w | to 100 | mL |
| 97 | I-1 | 3 | g |
| | 1-Methoxy-2-propanol/glycerol carbonate | to 100 | mL |
| | 65:35% w/w | | |
| 98 | I-1 | 1.99 | g |
| | 1-Methoxy-2-propanol/glycerol carbonate | to 100 | mL |
| | 65:35% w/w | | |
| 99 | I-1 | 3 | g |
| | 1-Methoxy-2-propanol/propylene carbonate | to 100 | mL |
| | 65:35% w/w | | |
| 100 | I-1 | 1.99 | g |
| | 1-Methoxy-2-propanol/propylene carbonate | to 100 | mL |
| | 65:35% w/w | | |
| 101 | I-2 | 1.99 | g |
| | Isopropanol/propylene carbonate 65:35% w/w | to 100 | mL |
| 102 | I-3 | 3 | g |
| | Isopropanol/propylene carbonate 65:35% w/w | to 100 | mL |
| 103 | I-3 | 1.99 | g |
| | Isopropanol/propylene carbonate 65:35% w/w | to 100 | mL |
| 104 | I-4 | 3 | g |
| | Isopropanol/propylene carbonate 65:35% w/w | to 100 | mL |
| 105 | I-4 | 1.99 | g |
| | Isopropanol/propylene carbonate 65:35% w/w | to 100 | mL |
| 106 | I-1 | 4.5 | g |
| | Isopropanol/propylene carbonate 25:75% w/w | to 100 | mL |
| 107 | I-1 | 1.5 | g |
| | Isopropanol/propylene carbonate 25:75% w/w | to 100 | mL |

The formulations with numbers 1 to 65 were produced and good solubilities and complete dissolution of the active ingredient was observed.

The experiments with numbers 66 to 107 are further hypothetical examples in which, based on the solubility experiments that were conducted, the actual examples prepared and based on the chemical structure of the active ingredients and the solvent selected, the desired solubility can be predicted.

### EXAMPLES OF IN VITRO AND IN VIVO TESTS

Further properties and efficacy of formulations according to the invention are described below.

### Example 1

Determination of the Spreading Properties of Colored Placebo Formulations on Cattle Skin

The aim of this in vitro study is the determination of the spreading properties of colored placebo formulations, i.e. without active ingredient, on cattle skin. The results shown with these tests can be will apply to spreading effects of formulations with an active ingredient. The tests were carried out with a dual solvent phase consisting of isopropanol (IPA) and propylene carbonate (PPC).

For this purpose, pieces of epidermis with the lightest colored coat as possible of about 10×10 cm were obtained from cattle after slaughter and placed on a hotplate covered with aluminum. The plate was heated to 34° C.+/−0.5° C. Skin pieces of shorn skin and with natural hair length were examined. 0.025% Brilliant Blue FCF was added as colorant. The behavior of the formulations on the skin surface and the spread area were measured over 60 minutes. The spread was additionally assessed by a rating system. In this case, such formulations were particularly advantageous and suitable for later use on the animal, because the spreading properties of which within the observation period achieved sufficient distribution on the skin piece.

The results are listed in Table 7, which shows the spreading characteristics of colored placebo formulations on skin pieces.

Table 7 shows the area which the formulation occupies on a skin sample directly after application and after one hour, and also shows the volumes of formulation applied, the hair length of the hair on the skin and the temperature of the skin.

TABLE 7

| | | | | Surface | Area [cm²] | Area [cm²] |
|---|---|---|---|---|---|---|
| Formulation | | Volumes | Hair | temperature | (0 min, after | (1 h after |
| (see Tab. 8) | IPA:PPC | [μl] | length | [° C.] | application) | application) |
| i | 1: 0:100 | 250 | shaved | 34 | 1.445 | 1.963 |
| ii | 2; 25:75 | 250 | shaved | 34 | 6.872 | 7.846 |
| iii | 3; 50:50 | 250 | shaved | 34 | 12.566 | 12.566 |
| iv | 4; 65:35 | 250 | shaved | 34 | 14.184 | 15.904 |
| v | 5; 75:25 | 250 | shaved | 34 | 14.137 | 15.708 |
| vi | 6; 90:10 | 250 | shaved | 34 | 12.566 | 19.635 |
| vii | 7; 100:0 | 250 | shaved | 34 | 7.069 | 15.504 |
| i. | 1: 0:100 | 250 | unshaved | 34 | 1.319 | 1.374 |
| ii. | 2; 25:75 | 250 | unshaved | 34 | 3.534 | 3.789 |
| iii. | 3; 50:50 | 250 | unshaved | 34 | 2.827 | 3.471 |
| iv. | 4; 65:35 | 250 | unshaved | 34 | 6.480 | 7.265 |
| v. | 5; 75:25 | 250 | unshaved | 34 | 2.356 | 2.937 |
| vi | 6; 90:10 | 250 | unshaved | 34 | 10.603 | 19.439 |
| vii | 7; 100:0 | 250 | unshaved | 34 | 3.927 | 4.909 |

It is evident from the present table that the spreading characteristics are particularly advantageous. The best results were achieved with a ratio of isopropanol (IPA) to propylene carbonate (PPC) of 65:35.

Although the test also shows a good result with a ratio of isopropanol (IPA) to propylene carbonate (PPC) of 90:10, this solvent phase had poorer solubility compared to the formulation according to the invention.

TABLE 8

| | | Ratio of isopropanol |
|---|---|---|
| Number of the | Brilliant Blue | (IPA):propylene |
| formulation | FCF colorant | carbonate (PPC) |
| I | 0.025% w/w | 0:100 |
| Ii | 0.025% w/w | 25:75 |
| Iii | 0.025% w/w | 50:50 |
| Iv | 0.025% w/w | 65:35 |
| V | 0.025% w/w | 75:25 |
| Vi | 0.025% w/w | 90:10 |
| Vii | 0.025% w/w | 100:0 |

### Example 2

In example 2, the spreading and run-off properties of colored placebo formulations on cattle skin was determined. The aim of this in vitro study was the determination of the spreading and run-off properties of colored placebo formulations on cattle skin. 0.025% Brilliant Blue FCF was added as colorant.

The formulations used correspond to those in Table 8.

For this purpose, pieces of hide with the lightest colored coat as possible of about 10×10 cm were obtained from cattle after slaughter and placed on a hotplate covered with aluminum. The plate was heated to 34+/−0.5° C. For the run-off test, the hotplates were set at an angle of 45°+/−2°. A piece of filter paper was secured beneath the skin piece.

The behavior of the formulations on the skin surface and the run-off rate were recorded on video over one minute and the run-off rate then calculated by means of a straightedge ruler. The spread was assessed by a rating system.

In this case, the formulations were particularly suitable for later use on the animal, because the run-off properties within the observation period resulted in no, or only minimal, coloration of the filter paper. The formulations also achieved sufficient distribution on the skin piece.

The results are shown in Table 9, which measures the run-off rate of the longest run trace over 1 minute after application of 750 μl of colored placebo formulation.

TABLE 9

| | | Natural | | Rate of | | | |
|---|---|---|---|---|---|---|---|
| | | variation | | longest | | | |
| | | of hair | | run trace | | Pass through | |
| IPA:PPC | Formulation No. | length | Behavior | [mm/sec] | | of filter | Surface distribution |
| 0:100 | I | mid- | Remains on the hair | 85.0 | | Starts after 2 | Application strip remains |
| | | length | surface and runs off the | | | sec and drips | narrow (<1 cm) but |
| | | | surface in 3 narrow | | | intensively | sinks into the coat |
| | | | tracks | | | thereafter | somewhat from the |
| | | | | | | | surface |
| 25:75 | ii | mid- | Sinks into the coat | 10.0 | | Starts after 40 | Application strip remains |
| | | length | forming 2 narrow run | | | sec and | narrow (<2 cm), runs off |
| | | | tracks which clearly | | | thereafter drips, | the skin, not on the coat. |
| | | | visibly run down and | | | dripped off | |
| | | | combine at the bottom | | | amounts less | |
| | | | | | | than 0:100 | |
| 50:50 | iii | Long | Rapidly sinks into the | 0.8 | | Filter after 9 | Application strip remains |
| | | | coat and a blue sheen is | | | mins has blue | narrow (<2 cm), runs off |
| | | | visible under the coat - | | | drops but | the skin, not on the coat. |
| | | | there are no sharp run | | | amount less | |
| | | | tracks rather surface | | | than 25:72 | |
| | | | spreading | | | | |
| 65:35 | iv | mid- | Rapidly sinks into the | 1.3 | area | Filter after 7.22 | Application strip |
| | | length | coat and a blue sheen is | | | sec with a small | broadens (2-4 cm), runs |
| | | | visible under the coat - | | | n blue point | down the surface of the |
| | | | there are no narrow run | | | | skin |
| | | | tracks - extensive | | | | |
| | | | spreading | | | | |
| 75:25 | v | Long | Line is not broad - | 3.3 | [area] | No pass through | Application strip remains |
| | | | relatively broad | | | of filter/dripping | narrow (<1 cm), run |
| | | | downward running thin | | | off | tracks run at the start |
| | | | tracks | | | | then narrow |
| 90:10 | vi | mid- | rapid very flat with run | 1.7 | area | No pass through | Application strip |
| | | length | tracks | | | of filter/dripping | broadens upwards and |
| | | | | | | off | downwards (4 cm) short |
| | | | | | | | run tracks run down on |
| | | | | | | | the skin surface |
| 100:0 | vii | Long | Rapidly distributes over | 2.7 | area | No pass through | Application strip appears |
| | | | large area with broad | | | of filter/dripping | narrow on the surface - |
| | | | run tracks | | | off | rapid distribution on the |
| | | | | | | | skin into an area with 3 |
| | | | | | | | visible short run tracks |

It is evident in turn from these tests that the test with the claimed proportions of isopropanol (IPA) and propylene carbonate (PPC) have afforded very good results since run-off properties here within the observation period resulted in no, or only minimal, coloration of the filter paper and in this case achieved sufficient distribution on the skin piece.

### Example 3

In example 3, the efficacy of pour-on formulations against horn flies (Haematobia irritans irritans) on cattle was determined. The aim of the study was the determination of the prophylactic efficacy of insecticidal formulations on experimentally generated Haematobia irritans infestations in cattle.

The study was carried out as a blind, negative controlled, randomized parallel group efficacy study. Each group comprised 5 animals. In advance by fly counting, all animals prior to treatment were sorted into a descending sequence and then grouped into 4 (x) blocks) of 5 animals. Cattle were randomly assigned to the blocks and sorted into ascending number sequence. The cattle were installed tethered individually in gauze-enclosed spaces. Each animal was infested with ca. 200 unfed flies on day −5, 1, 7, 14, 21, 28 and in some cases also on day 35 and 42. The number of flies on the cattle was determined on day 1 four hours after infestation, 24 h after infestation on day 2, on days 8, 15, 22, 29, and if appropriate on day 36, 43, 50 and 57. The efficacy against fly infestation is determined by comparing the number of flies on treated cattle with the number of flies on untreated cattle. In this case, the treatment was judged as successful if an at least 90% effect was measured for the treatment group compared to the control group.

The results are shown in Table 10, which illustrates the effect of formulations according to the invention on small horn flies (Haematobia irritans irritans) on cattle.

TABLE 10

| Test | Formulation | Dose | | | | | | | | | |
|---|---|---|---|---|---|---|---|---|---|---|---|
| block | example | mg/kg | dpt 2 | dpt 8 | dpt 15 | dpt 22 | dpt 29 | dpt 36 | dpt 43 | dpt 50 | dpt 57 |
| C | 5 | 0.5 | >90 | >90 | >90 | >90 | >90 | >90 | <90 | <90 | <90 |
| C | 7 | 1 | >90 | >90 | >90 | >90 | >90 | >90 | >90 | <90 | <90 |
| B | 1 | 1.5 | >90 | >90 | >90 | >90 | >90 | >90 | >90 | na | na |
| C | 8 | 2 | >90 | >90 | >90 | >90 | >90 | >90 | >90 | >90 | >90 |
| C | 9 | 4 | >90 | >90 | >90 | >90 | >90 | >90 | >90 | >90 | >90 |
| B | 3 | 4.5 | >90 | >90 | >90 | >90 | >90 | >90 | >90 | na | na |
| A | 15 | 1.5 | >90 | >90 | >90 | >90 | <90 | <90 | na | na | na |
| A | 16 | 4.5 | >90 | >90 | >90 | >90 | <90 | <90 | na | na | na |
| A | 13 | 3 | >90 | >90 | >90 | >90 | >90 | <90 | na | na | na |
| B | 11 | 3 | >90 | >90 | >90 | >90 | >90 | >90 | >90 | na | na |

Dpt = days post treatment
na = no data

It is evident from Table 10 that examples 5 and 7, even at low doses, have a long-lasting and effective action. Further, examples 8, 9, 1, 3 and 11 are also effective long-term.

### Example 4

In example 4, the efficacy of pour-on formulations against ticks (Rhipicephalus (Boophilus) microplus) on cattle was determined.

The aim of the study was the determination of therapeutic and prophylactic efficacy of acaricidal formulations on experimentally generated Rhipicephalus (Boophilus) microplus infestations in cattle.

The study was carried out as a parallel group, unblinded, negative-controlled, randomized efficacy study. Each group comprised 5 animals. In advance by sex and individual tick count of infestations prior to treatment (day−14 to −2), all animals were sorted in decreasing sequence and then grouped into 2 (x) blocks) of 5 animals. Cattle were randomly assigned to the blocks and sorted into ascending number sequence. Each animal was infested with 3000 larvae on day−35, −33, −29. −27, −22, −20, −15, −13, −8, −6, −1, 5, 7, 12, 14, 19, 21, 26 and 28. Engorged females dropping off were collected daily on days −14 to −2 and 0-56. Ticks from −14 to −2 were used for the grouping, ticks from day 1−28 for determination of the therapeutic effect, and ticks from day 29 up to the end of the tick falling phase were used for determination of the prophylactic effect. The fertility of the ticks was also determined. This was accomplished by assessment of oviposition and larvae hatch of the laid eggs from mixed samples of the engorged female ticks. In this case, >60% of larvae hatch signifies normal hatch rate. The compounds tested achieved here no significant additional effect.

For therapeutic efficacy against existing tick infestations, the treatment was judged as successful when a 90% effect (based on the number of engorged female ticks falling off on days 1-28 after treatment) for the treatment group was measured compared to the control group. Accordingly, no efficacy was present if the effect is below 90%.

For prophylactic efficacy against newly emerging tick infestations, the treatment was judged as successful when a 90% effect (based on the number of engorged female ticks falling off on days 29-56 after treatment) for the treatment group was measured compared to the control group. Accordingly, no sufficient efficacy is shown if the effect was below 90%.

The results are shown in Tables 11 and 12. Table 11 shows the therapeutic effect of formulations according to the invention on cattle on all stages of the cattle tick (Rhipicephalus (Boophilus) microplus) and Table 12 shows the prophylactic effect of formulations according to the invention on cattle on emerging cattle tick (Rhipicephalus (Boophilus) microplus) infestations.

TABLE 11

| Test | Formulation | Dose | Study day after treatment | | | | | | | | | | | | |
|---|---|---|---|---|---|---|---|---|---|---|---|---|---|---|---|
| block | example | mg/kg | 1 | 2 | 3 | 4 | 5 | 6 | 7 | 8 | 9 | 10 | 11 | 12 | 13 |
| C | 7 | 1 | na | na | >90 | >90 | >90 | >90 | >90 | >90 | >90 | >90 | >90 | >90 | >90 |
| C | 8 | 2 | na | na | >90 | >90 | >90 | >90 | >90 | >90 | >90 | >90 | >90 | >90 | >90 |
| C | 9 | 4 | na | na | >90 | >90 | >90 | >90 | >90 | >90 | >90 | >90 | >90 | >90 | >90 |
| A | 15 | 1.5 | >90 | >90 | >90 | >90 | >90 | >90 | >90 | >90 | >90 | >90 | >90 | >90 | >90 |
| A | 16 | 4.5 | >90 | >90 | >90 | >90 | >90 | >90 | >90 | >90 | >90 | >90 | >90 | >90 | >90 |
| A | 13 | 3 | >90 | >90 | >90 | >90 | >90 | >90 | >90 | >90 | >90 | >90 | >90 | >90 | >90 |
| B | 2 | 2 | >90 | >90 | >90 | >90 | >90 | >90 | >90 | >90 | >90 | >90 | >90 | >90 | >90 |
| B | 12 | 2 | >90 | >90 | >90 | >90 | >90 | >90 | >90 | >90 | >90 | >90 | >90 | >90 | >90 |

| Test | Study day after treatment | | | | | | | | | | | | | | |
|---|---|---|---|---|---|---|---|---|---|---|---|---|---|---|---|
| block | 14 | 15 | 16 | 17 | 18 | 19 | 20 | 21 | 22 | 23 | 24 | 25 | 26 | 27 | 28 |
| C | >90 | >90 | >90 | >90 | >90 | >90 | >90 | >90 | >90 | >90 | >90 | >90 | >90 | >90 | >90 |
| C | >90 | >90 | >90 | >90 | >90 | >90 | >90 | >90 | >90 | >90 | >90 | >90 | >90 | >90 | >90 |
| C | >90 | >90 | >90 | >90 | >90 | >90 | >90 | >90 | >90 | >90 | >90 | >90 | >90 | >90 | >90 |
| A | >90 | >90 | >90 | >90 | >90 | >90 | >90 | >90 | >90 | >90 | >90 | >90 | >90 | >90 | >90 |
| A | >90 | >90 | >90 | >90 | >90 | >90 | >90 | >90 | >90 | >90 | >90 | >90 | >90 | >90 | >90 |
| A | >90 | >90 | >90 | >90 | >90 | >90 | >90 | >90 | >90 | >90 | >90 | >90 | >90 | >90 | >90 |
| B | >90 | >90 | >90 | >90 | >90 | >90 | >90 | >90 | >90 | >90 | >90 | >90 | >90 | >90 | >90 |
| B | >90 | >90 | >90 | >90 | >90 | >90 | >90 | >90 | >90 | >90 | >90 | >90 | >90 | >90 | >90 |

na = no data

TABLE 12

| Test | Formulation | Dose | Study day after treatment | | | | | | | | | | | | |
|---|---|---|---|---|---|---|---|---|---|---|---|---|---|---|---|
| block | example | mg/kg | 29 | 30 | 31 | 32 | 33 | 34 | 35 | 36 | 37 | 38 | 39 | 40 | 41 |
| C | 7 | 1 | >90 | >90 | >90 | >90 | >90 | >90 | >90 | >90 | >90 | >90 | >90 | >90 | >90 |
| C | 8 | 2 | >90 | >90 | >90 | >90 | >90 | >90 | >90 | >90 | >90 | >90 | >90 | >90 | >90 |
| C | 9 | 4 | >90 | >90 | >90 | >90 | >90 | >90 | >90 | >90 | >90 | >90 | >90 | >90 | >90 |
| A | 15 | 1.5 | >90 | >90 | >90 | >90 | >90 | >90 | >90 | >90 | >90 | >90 | >90 | >90 | >90 |
| A | 16 | 4.5 | >90 | >90 | >90 | >90 | >90 | >90 | >90 | >90 | >90 | >90 | >90 | >90 | >90 |
| A | 13 | 3 | >90 | >90 | >90 | >90 | >90 | >90 | >90 | >90 | >90 | >90 | >90 | >90 | >90 |
| B | 2 | 2 | >90 | >90 | >90 | >90 | >90 | >90 | >90 | >90 | >90 | >90 | >90 | >90 | >90 |
| B | 12 | 2 | >90 | >90 | >90 | >90 | >90 | >90 | >90 | >90 | >90 | >90 | >90 | >90 | >90 |

| Test | Study day after treatment | | | | | | | | | | | | | | |
|---|---|---|---|---|---|---|---|---|---|---|---|---|---|---|---|
| block | 42 | 43 | 44 | 45 | 46 | 47 | 48 | 49 | 50 | 51 | 52 | 53 | 54 | 55 | 56 |
| C | >90 | >90 | >90 | >90 | >90 | >90 | >90 | >90 | >90 | >90 | >90 | >90 | na | >90 | na |
| C | >90 | >90 | >90 | >90 | >90 | >90 | >90 | >90 | >90 | >90 | >90 | >90 | na | >90 | na |
| C | >90 | >90 | >90 | >90 | >90 | >90 | >90 | >90 | >90 | >90 | >90 | >90 | na | >90 | na |
| A | >90 | >90 | >90 | >90 | >90 | >90 | >90 | >90 | >90 | >90 | >90 | >90 | >90 | na | na |
| A | >90 | >90 | >90 | >90 | >90 | >90 | >90 | >90 | >90 | >90 | >90 | >90 | >90 | na | na |
| A | >90 | >90 | >90 | >90 | >90 | >90 | >90 | >90 | >90 | >90 | >90 | >90 | >90 | na | na |
| B | >90 | >90 | >90 | >90 | >90 | >90 | >90 | >90 | >90 | >90 | >90 | >90 | >90 | >90 | na |
| B | >90 | >90 | >90 | >90 | >90 | >90 | >90 | >90 | >90 | >90 | >90 | >90 | >90 | >90 | na |

na = no data

In Tables 11 and 12, it can be seen that all active ingredients in the compositions according to the invention have high efficacy, even at low doses, both for prophylactic and therapeutic efficacy against tick infestations.

### Example 5

In accordance with example 5, the efficacy of pour-on formulations against hair lice (Bovicola bovis) on cattle was determined. The aim of the study was the determination of therapeutic and prophylactic efficacy of insecticidal formulations on naturally existing Bovicola bovis infestations in cattle.

The study was carried out as a parallel group, unblinded, negative-controlled efficacy study. Each group comprised 5 animals. Lice were counted on day −2 before treatment and on days 2, 14, 28 and 42. In this case, the hair coat of each animal was screened at 10 partings and all nymphal and adult hair lice stages were counted. The results were totaled for each animal, averaged between the animals of one group and compared with the numbers of hair lice of an untreated control. The acute efficacy against existing hair lice infestations was determined on day 2. In this case, the treatment was judged as successful if an at least 90% effect was measured for the treatment group compared to the control group. The persisting effectiveness of existing hair lice infestations was measured in the counts on day 14, 21 and 42.

In this case, the treatment was judged as successful if a 100% effect was measured for the treatment group compared to the control group. Accordingly, in the case of effects below 100%, treatment was judged unsuccessful.

TABLE 13

| Test | Formulation | Dose | | | | |
|---|---|---|---|---|---|---|
| block | example | mg/kg | dpt 2 | dpt 14 | dpt 28 | dpt 42 |
| B | 2 | 2 | >90 | 100 | 100 | 100 |
| B | 12 | 2 | >90 | 100 | 100 | 100 |
| A | 16 | 3 | <90 | <100 | <100 | 100 |
| A | 13 | 3 | <90 | <100 | 100 | 100 |
| A | 15 | 3 | <90 | <100 | 100 | 100 |

Dpt = days post treatment

Table 13 shows the effect of formulations according to the invention on hair lice (Bovicola bovis) on cattle.

It is evident from Table 13 that Examples 2 and 16 in particular have comparatively rapid high efficacy when they were used in a composition according to the invention with a defined solvent mixture.

It is therefore evident from the above that pharmaceutically active compositions according to the invention demonstrate good solubility for active ingredients of the class of benzamides and with these enable an effective active composition, especially for use in the treatment of or prophylaxis of parasite infestation, particularly in the case of non-human organisms. In this case, use as pour-on application is of particular advantage.

1. A pharmaceutical composition comprising at least one pharmaceutically active ingredient, which is soluble in a solvent phase, wherein the pharmaceutically active ingredient is a fluorinated heteroarylamide, and wherein the solvent phase is a solvent mixture comprising at least a first solvent and a second solvent different from the first solvent, wherein the first solvent comprises a C₁ to C₁₅-alcohol, wherein the second solvent comprises an organic carbonate, wherein the first solvent is present in a proportion from 50% by weight to 95% by weight and wherein the second solvent is present in a proportion from 5% by weight to 50% by weight, based on the sum total of the solvents in the solvent phase.

2. The pharmaceutical composition according to claim 1, wherein the first solvent is selected from C₁-C₄-alkanols, optionally substituted with at least one C₁-C₄-alkoxy group or phenyl group, and wherein the first solvent is selected from the group consisting of ethanol, n-propanol, isopropanol, butanol.

3. The pharmaceutical composition according to claim 1, wherein the second solvent is selected from aliphatic C₁-C₄-carbonates, optionally substituted by an hydroxyl group.

4. The pharmaceutical composition according to claim 1, wherein characterized in that the first solvent is isopropanol and the second solvent is propylene carbonate.

5. The pharmaceutical composition according to claim 1, wherein the solvent mixture consists of the first solvent and the second solvent.

6. The pharmaceutical composition according claim 1, wherein the solvent mixture further comprises a third solvent different from the first solvent and the second solvent in addition to the first solvent and the second solvent.

7. The pharmaceutical composition according to claim 6, wherein the third solvent is selected from the group consisting of water, butylhydroxytoluene, N-methylpyrrolidone, 2-pyrrolidone, dimethyl sulfoxide, triethyl phosphate, benzyl benzoate, octyldodecanol, paraffin, triglycerides, propylene glycol octanoate decanoate, glycol ether.

8. The pharmaceutical composition according to claim 1, wherein the second solvent is present at a content of ≥20% by weight to ≤45% by weight, optionally at a content of ≥30% by weight to ≤40% by weight, based on the sum total of the solvents in the solvent mixture.

9. The pharmaceutical composition according to claim 1, wherein the active ingredient is selected from one or more compounds of formula (I): 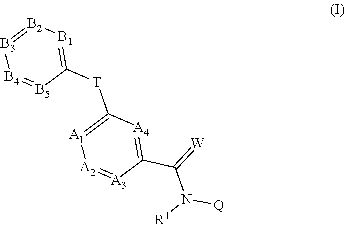
in which
R¹ is H, in each case optionally substituted C₂-C₆-alkenyl, C₂-C₆-alkynyl, C₃-C₇-cycloalkyl, C₁-C₆-alkylcarbonyl, C₁-C₆-alkoxycarbonyl, aryl-(C₁-C₃)-alkyl, heteroaryl-(C₁-C₃)-alkyl or is optionally substituted C₁-C₆-alkyl, optionally H or optionally C₁-C₂-alkyl, optionally H or methyl, optionally H,
the moieties are as follows:
A₁ is CR² or N,
A₂ is CR³ or N,
A₃ is CR⁴ or N,
A₄ is CR⁵ or N,
B₁ is CR⁶ or N,
B₂ iS CR⁷ or N,
B₃ is CR⁸ or N,
B₄ is CR⁹ or N, and
B₅ is CR¹⁰ or N,
but not more than three of the A₁ to A₄ moieties are N and not more than three of the B₁ to B₅ moieties are N;
R², R³, R⁴, R⁵, R⁶, R⁷, R⁹ and R¹⁰ are each independently of one another H, halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, N—C₁-C₆-alkylamino, N,N-di-C₁-C₆-alkylamino or N—C₁-C₃-alkoxy-C₁-C₄-alkylamino or 1-pyrrolidinyl;
if neither of the A₂ and A₃ moieties is N, R³ and R⁴ together with the carbon atom to which they are bonded may form a 5- or 6-membered ring containing 0, 1 or 2 nitrogen atoms and/or 0 or 1 oxygen atom and/or 0 or 1 sulfur atom; or
if neither of the A₁ and A₂ moieties is N, R² and R³ together with the carbon atom to which they are bonded may form a 6-membered ring containing 0, 1 or 2 nitrogen atoms;
R⁸ is halogen, cyano, nitro, in each case optionally substituted C₁-C₆-alkyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, N—C₁-C₆-alkylamino or N,N-di-C₁-C₆-alkylamino;
W is O or S,
Q is H, formyl, hydroxyl, amino or in each case optionally substituted C₁-C₆-alkyl, C₂-C₆-alkenyl, C₂-C₆-alkynyl, C₃-C₆-cycloalkyl, C₁-C₆-heterocycloalkyl, C₁-C₄-alkoxy, C₁-C₆-alkyl-C₃-C₆-cycloalkyl, C₃-C₆-cycloalkyl-C₁-C₆-alkyl, C₆-,C₁₀-C₁₄-aryl, C₁-C₅-heteroaryl, C₆-,C₁₀-,C₁₄-aryl-(C₁-C₃)-alkyl, C₁-C₆-heteroaryl-(C₁-C₃)-alkyl, N—C₁-C₄-alkylamino, N—C₁-C₄-alkylcarbonylamino, or N,N-di-C₁-C₄-alkylamino; or
is an optionally poly-V-substituted unsaturated 6-membered carbocycle; or
is an optionally poly-V-substituted unsaturated 4-, 5- or 6-membered heterocyclic ring, where
V independently of one another is halogen, cyano, nitro, in each case optionally substituted C₁-C₆-alkyl, C₁-C₄-alkenyl, C₁-C₄-alkynyl, C₃-C₆-cycloalkyl, C₁-C₆-alkoxy, N—C₁-C₆-alkoxyimino-C₁-C₃-alkyl, C₁-C₆-alkylsulfanyl, C₁-C₆-alkylsulfinyl, C₁-C₆-alkylsulfonyl, or N,N-di-(C₁-C₆-alkyl)amino;
T is an optionally substituted 5-membered heteroaromatic system containing not more than 2 heteroatoms (1 or 2 heteroatoms), optionally as four carbon atoms and one (1) heteroatom, optionally one (1) nitrogen, one (1) oxygen or one (1) sulfur atom or three carbon atoms and two heteroatoms, optionally two nitrogen atoms, one (1) nitrogen and one (1) oxygen atom, or one (1) nitrogen and one (1) sulfur atom,
And/or a salt, N-oxide and/or a tautomeric form of one or more compounds of the (I).

10. The pharmaceutical composition according to claim 9, wherein the active ingredient is selected from the group consisting of
2-chloro-N-cyclopropyl-5-[1-[2,6-dichloro-4-[1,2,2,2-tetrafluoro-1-(trifluoromethyl)-ethyl]phenyl]pyrazol-4-yl]-N-methylpyridine-3-carboxamide 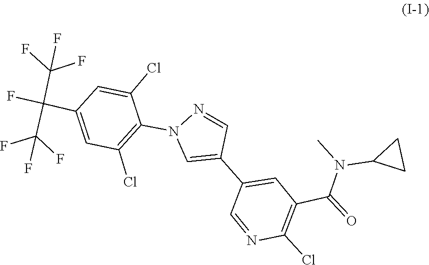 [SMILES: CN(C(=O)c1cc(-c2cnn(-c3c(Cl)cc(C(F)(C(F)(F)F)C(F)(F)F)cc3Cl)c2)cnc1Cl)C1CC1]
2-chloro-N-cyclopropyl-5-[1-[2-chloro-4-[1,2,2,2-tetrafluoro-1-(trifluoromethyl)ethyl]-6-(trifluoromethoxy)phenyl]-1H-pyrazol-4-yl]-3-pyridinecarboxamide: 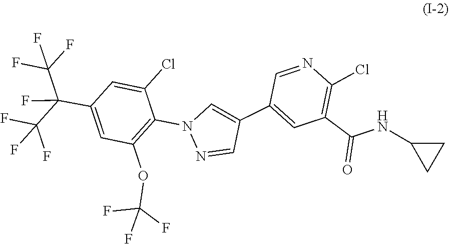 [SMILES: O=C(NC1CC1)c1cc(-c2cnn(-c3c(Cl)cc(C(F)(C(F)(F)F)C(F)(F)F)cc3OC(F)(F)F)c2)cnc1Cl]
2-chloro-5-[1-[2-chloro-4-[1,2,2,2-tetrafluoro-1-(trifluoromethyl)ethyl]-6-(trifluoromethyl)phenyl]-1H-pyrazol-4-yl]-N-cyclopropyl-3-pyridinecarboxamide: 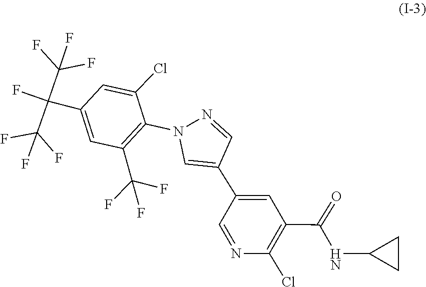 [SMILES: O=C(NC1CC1)c1cc(-c2cnn(-c3c(Cl)cc(C(F)(C(F)(F)F)C(F)(F)F)cc3C(F)(F)F)c2)cnc1Cl]
2-chloro-5-[1-[2-bromo-4-[1,2,2,2-tetrafluoro-1-(trifluoromethyl)ethyl](trifluoromethoxy)phenyl]-1H-pyrazol-4-yl]-N-cyclopropylpyridine-3-carboxamide 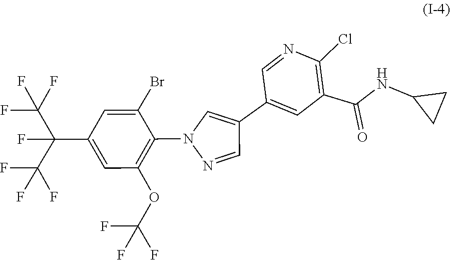 [SMILES: O=C(NC1CC1)c1cc(-c2cnn(-c3c(Br)cc(C(F)(C(F)(F)F)C(F)(F)F)cc3OC(F)(F)F)c2)cnc1Cl]
2-chloro-N-cyclopropyl-5-[1-[2,6-dichloro-4-[1,2,2,2-tetrafluoro-1-(trifluoromethyl)ethyl]phenyl]pyrazol-4-yl]pyridine-3-carboxamide 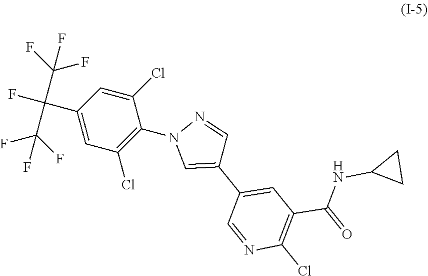 [SMILES: O=C(NC1CC1)c1cc(-c2cnn(-c3c(Cl)cc(C(F)(C(F)(F)F)C(F)(F)F)cc3Cl)c2)cnc1Cl]
2-chloro-N-cyanocyclopropyl-5-[1-[2-chloro-4-[1,2,2,2-tetrafluoro-1-(trifluoromethyl)ethyl]-6-(trifluoromethoxy)phenyl]-1H-pyrazol-4-yl]benzamide: 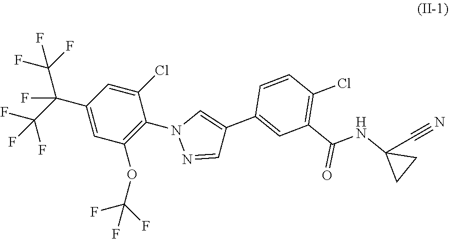 [SMILES: N#CC1(NC(=O)c2cc(-c3cnn(-c4c(Cl)cc(C(F)(C(F)(F)F)C(F)(F)F)cc4OC(F)(F)F)c3)ccc2Cl)CC1]
2-chloro-5-[1-[2-chloro-4-[1,2,2,2-tetrafluoro-1-(trifluoromethyl)ethyl]-6-(trifluoromethoxy)phenyl]pyrazol-4-yl]-N-cyclopropylbenzamide 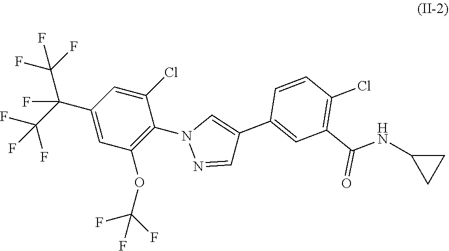 [SMILES: O=C(NC1CC1)c1cc(-c2cnn(-c3c(Cl)cc(C(F)(C(F)(F)F)C(F)(F)F)cc3OC(F)(F)F)c2)ccc1Cl]
5-[1-[2-bromo-4-[1,2,2,2-tetrafluoro-1-(trifluoromethyl)ethyl]-6-(trifluoromethoxy)phenyl]pyrazol-4-yl]-2-chloro-N-cyclopropylbenzamide 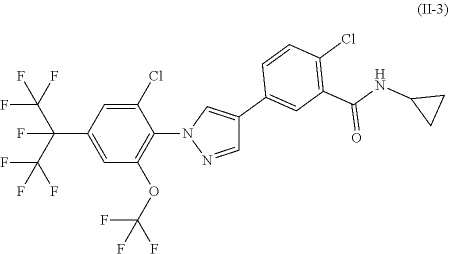 [SMILES: O=C(NC1CC1)c1cc(-c2cnn(-c3c(Cl)cc(C(F)(C(F)(F)F)C(F)(F)F)cc3OC(F)(F)F)c2)ccc1Cl]
2-chloro-5-[1-[2-chloro-4-[1,2,2,2-tetrafluoro-1-(trifluoromethyl)ethyl]-6-(trifluoromethyl)phenyl]pyrazol-4-yl]-N-cyclopropylbenzamide 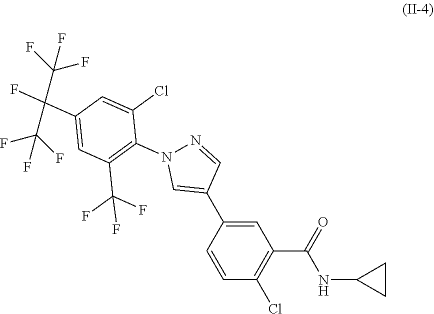 [SMILES: O=C(NC1CC1)c1cc(-c2cnn(-c3c(Cl)cc(C(F)(C(F)(F)F)C(F)(F)F)cc3C(F)(F)F)c2)ccc1Cl]
2-chloro-N-cyclopropyl-5-[1-[2,6-dichloro-4-[1,2,2,2-tetrafluoro-1-(trifluoromethyl)ethyl]phenyl]pyrazol-4-yl]benzamide 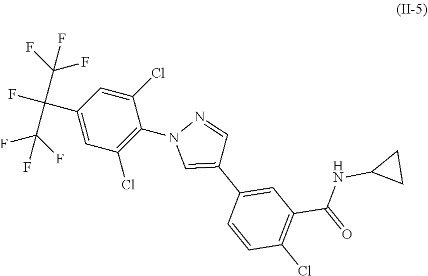 [SMILES: O=C(NC1CC1)c1cc(-c2cnn(-c3c(Cl)cc(C(F)(C(F)(F)F)C(F)(F)F)cc3Cl)c2)ccc1Cl]
2-chloro-N-cyclopropyl-5-[1-[2,6-dichloro-4-[1,2,2,2-tetrafluoro-1-(trifluoromethyl)ethyl]phenyl]pyrazol-4-yl]-N-methylbenzamide 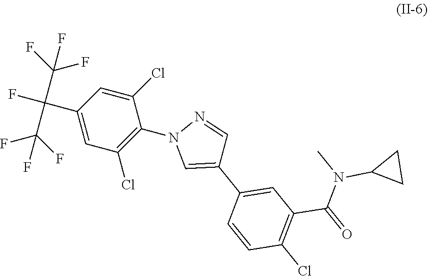 [SMILES: CN(C(=O)c1cc(-c2cnn(-c3c(Cl)cc(C(F)(C(F)(F)F)C(F)(F)F)cc3Cl)c2)ccc1Cl)C1CC1]
2-chloro-N-cyclopropyl-5-[1-[4-(1,1,1,2,3,3,3-heptafluoropropan-2-yl)-2-methyl-6-(trifluoromethyl)phenyl]-1H-pyrazol-4-yl]benzamide: 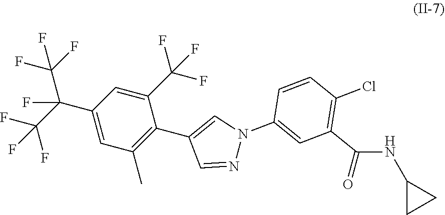 [SMILES: Cc1cc(C(F)(C(F)(F)F)C(F)(F)F)cc(C(F)(F)F)c1-c1cnn(-c2ccc(Cl)c(C(=O)NC3CC3)c2)c1]
2-chloro-N-(1-cyanocyclopropyl)-5-[1-[4-(1,1,1,2,3,3,3-heptafluoropropan-2-yl)-2-methyl-6-(trifluoromethyl)phenyl]-1H-pyrazol-4-yl]benzamide: 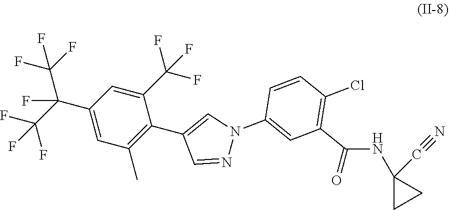 [SMILES: Cc1cc(C(F)(C(F)(F)F)C(F)(F)F)cc(C(F)(F)F)c1-c1cnn(-c2ccc(Cl)c(C(=O)NC3(C#N)CC3)c2)c1]
2-chloro-N-cyclopropyl-5-[4-[2,6-dimethyl-4-[1,2,2,2-tetrafluoro-1-(trifluoromethyl)ethyl]phenyl]pyrazol-1-yl]benzamide: 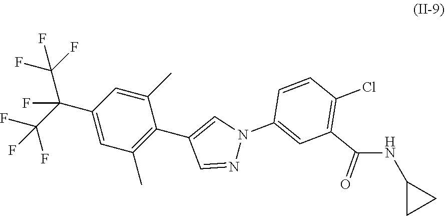 [SMILES: Cc1cc(C(F)(C(F)(F)F)C(F)(F)F)cc(C)c1-c1cnn(-c2ccc(Cl)c(C(=O)NC3CC3)c2)c1]
2-chloro-N-(1-cyanocyclopropyl)-5-[4-[2,6-dichloro-4-[1,2,2,2-tetrafluoro-1-(trifluoromethyl)ethyl]phenyl]pyrazol-1-yl]benzamide: 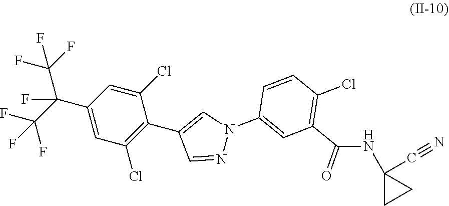 [SMILES: N#CC1(NC(=O)c2cc(-n3cc(-c4c(Cl)cc(C(F)(C(F)(F)F)C(F)(F)F)cc4Cl)cn3)ccc2Cl)CC1]
2-chloro-5-[3-[2-chloro-4-[1,2,2,2-tetrafluoro-1-(trifluoromethyl)ethyl](trifluoromethyl)phenyl]isoxazol-5-yl]-N-cyclopropylbenzamide: 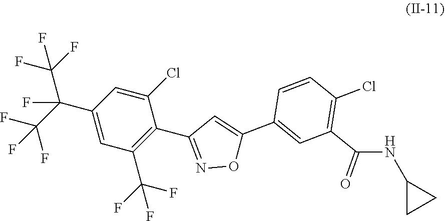 [SMILES: O=C(NC1CC1)c1cc(-c2cc(-c3c(Cl)cc(C(F)(C(F)(F)F)C(F)(F)F)cc3C(F)(F)F)no2)ccc1Cl]
2-chloro-N-(1-cyanocyclopropyl)-5-[3-[2-methyl-4-[1,2,2,2-tetrafluoro-1-(trifluoromethyl)ethyl]-6-(trifluoromethyl)phenyl]isoxazol-5-yl]benzamide: 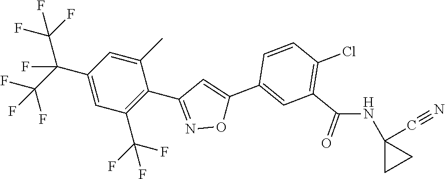 [SMILES: Cc1cc(C(F)(C(F)(F)F)C(F)(F)F)cc(C(F)(F)F)c1-c1cc(-c2ccc(Cl)c(C(=O)NC3(C#N)CC3)c2)on1]
2-chloro-N-(1-cyanocyclopropyl)-5-[1-[2,6-dichloro-4-[1,2,2,2-tetrafluoro-1-(trifluoromethyl)ethyl]phenyl]pyrrol-3-yl]benzamide: 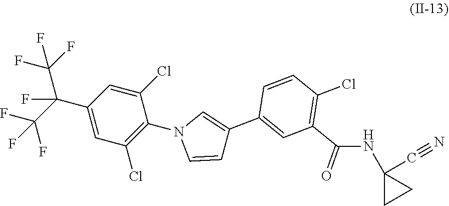 [SMILES: N#CC1(NC(=O)c2cc(-c3ccn(-c4c(Cl)cc(C(F)(C(F)(F)F)C(F)(F)F)cc4Cl)c3)ccc2Cl)CC1]
2-chloro-5-[3-[2-chloro-4-[1,2,2,2-tetrafluoro-1-(trifluoromethyl)ethyl](trifluoromethyl)phenyl]pyrrol-1-yl]-N-cyclopropylbenzamide:

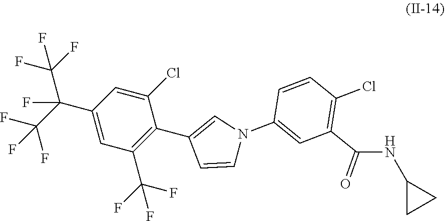 [SMILES: O=C(NC1CC1)c1cc(-n2ccc(-c3c(Cl)cc(C(F)(C(F)(F)F)C(F)(F)F)cc3C(F)(F)F)c2)ccc1Cl]

11. The pharmaceutical composition according to claim 1, wherein the pharmaceutically active ingredient is present in an amount of greater than or equal to 0.1% by weight to less than or equal to 5% by weight.

12. The pharmaceutical composition according to claim 1, for use in treatment of or prophylaxis of parasite infestation.

13. The pharmaceutical composition according to claim 12 for use in treatment of or prophylaxis of ectoparasite infestation.

14. The pharmaceutical composition according to claim 1 for use in treatment of or prophylaxis of parasite infestation in non-human organisms.

15. The pharmaceutical composition according to claim 14 for use in treatment of or prophylaxis of parasite infestation in one or more non-human organisms by dripping or pouring the composition onto the non-human organism.
